(19) United States
(12) Patent Application Publication
(10) Pub. No.: US 2012/0189613 A1
(43) Pub. Date: Jul. 26, 2012

(54) STIM2-MEDIATED CAPACITIVE CALCIUM ENTRY

(75) Inventors: Bernhard Nieswandt, Eibelstadt (DE); Guido Stoll, Rimpar (DE)

(73) Assignee: JULIUS-MAXIMILIANS-UNIVERSITÄT WÜRZBURG, Würzburg (DE)

(21) Appl. No.: 13/375,202

(22) PCT Filed: May 28, 2010
(86) PCT No.: PCT/EP10/57443

§ 371 Date: Apr. 2, 2012

(30) Foreign Application Priority Data
May 29, 2009 (EP) 09007194.5

(51) Int. Cl. A61K 39/395; A61K 38/18; C12Q 1/68; G01N 33/53; G01N 21/64; A61P 25/18; A61P 25/02; A61P 25/00; A61P 21/00; A61P 25/08; A61P 25/14; A61K 31/7088; A61P 25/28

(52) U.S. Cl. 424/130.1; 514/44 R; 514/44 A; 514/8.3; 435/6.12; 436/501; 436/63

(57) ABSTRACT

The present invention relates to a pharmaceutical composition comprising an inhibitor of STIM2 or an inhibitor of STIM2-regulated plasma membrane calcium channel activity and optionally a pharmaceutically acceptable carrier, excipient and/or diluent. Furthermore, the present invention relates to an inhibitor of STIM2 or an inhibitor of STIM2-regulated plasma membrane calcium channel activity for the treatment and/or prevention of a neurological disorder associated with pathologically increased cytosolic calcium concentrations. Also disclosed are methods of treating and/or preventing a neurological disorder associated with pathologically increased cytosolic calcium concentrations comprising administering a pharmaceutically effective amount of an inhibitor of STIM2 or of an inhibitor of STIM2-regulated plasma membrane calcium channel activity to a subject in need thereof. The present invention further relates to methods of identifying a compound suitable as a lead compound and/or as a medicament for the treatment and/or prevention of a neurological disorder associated with pathologically increased cytosolic calcium concentrations.

### CROSS-REFERENCE TO RELATED APPLICATION

This application is a 371 application that claims priority to PCT application EP2010/057443 filed on May 28, 2010, which is incorporated herein by reference in its entirety for all purposes.

### FIELD

The present invention relates to a pharmaceutical composition comprising an inhibitor of stromal interaction molecule 2 (STIM2) or an inhibitor of STIM2-regulated plasma membrane calcium channel activity, in particular an inhibitor of ORAI2 or ORAI3 (also designated as CRACM2 or CRACM3), and optionally a pharmaceutically acceptable carrier, excipient and/or diluent. Furthermore, the present invention relates to an inhibitor of STIM2 or an inhibitor of STIM2-regulated plasma membrane calcium channel activity for use in the treatment and/or prevention of a neurodegenerative disorder associated with pathologically increased cytosolic calcium concentrations. The present invention also relates to a method of treating and/or preventing a neurodegenerative disorder associated with pathologically increased cytosolic calcium concentrations comprising administering a pharmaceutically effective amount of an inhibitor of STIM2 or of an inhibitor of STIM2-regulated plasma membrane calcium channel activity to a subject in need thereof. The present invention further relates to methods of identifying a compound suitable as a lead compound and/or as a medicament for the treatment and/or prevention of a neurodegenerative disorder associated with pathologically increased cytosolic calcium concentrations.

### BACKGROUND

The intracellular Ca²⁺ concentration [Ca²⁺], is a major determinant of the physiological state in all eukaryotic cells (Berridge, 2003). In neurons, Ca²⁺ signals derived from intracellular stores or extracellular space are essential for fundamental functions, including synaptic transmission and plasticity. On the other hand, excessive cytosolic Ca²⁺ accumulation, i.e. a “calcium overload” such as observed under pathological conditions, can induce neuronal cell death. The mechanisms underlying this calcium overload induced neuronal cell death are poorly understood (Lipton, 1999; Berridge, 1998; Wojda, 2008; Mattson, 2007).

Two principal types of Ca²⁺ channels are firmly established to mediate Ca²⁺ entry into neurons: voltage-operated Ca²⁺ channels (VOCCs) and ionotropic receptor-operated (ligand-gated) channels (ROCs), including N-methyl-D-aspartate receptors (NMDARs) and some α-amino-3-hydroxy-5-methyl-isoxazole-4-propionate acid receptors (AMPARs)—that are activated by the excitatory neurotransmitter glutamate. Glutamate excitotoxicity is a well studied mechanism contributing to “calcium overload” and subsequent neurodegeneration in ischemia (Wojda, 2008; Burnashev, 2005; Rao, 2007).

In contrast, very little is known about plasma membrane Ca²⁺ channels (also called store-operated Ca²⁺ (SOC) channels) which are activated in response to Ca²⁺ store depletion to allow capacitive Ca²⁺ entry (CCE—also referred to as store-operated Ca²⁺ entry, SOCE) and store replenishment (Putney, 2003). In non-excitable cells, CCE is controlled by the endoplasmic reticulum (ER)-resident Ca²⁺ sensor STIM1, whereas the closely related STIM2 has been proposed to regulate basal cytosolic and ER Ca²⁺ concentrations with only a minor contribution to CCE.

Wojda et al. 2008 reviews the role of calcium ions in neuronal degeneration. The authors also summarize compounds that are currently being tested as potential neuroprotective agents. So far, the only compounds qualified for clinical trials are compounds acting on either glutamate receptors or voltage-operated calcium channels.

### SUMMARY

The technical problem underlying the present invention is the provision of alternative and/or improved means and methods for successfully treating neurodegenerative disorders associated with pathologically increased cytosolic calcium concentrations that form the basis or may allow for the development of more satisfactory medicaments for the treatment and/or prevention of these diseases.

The present invention relates to a pharmaceutical composition comprising an inhibitor of stromal interaction molecule 2 (STIM2) or an inhibitor of STIM2-regulated plasma membrane calcium channel activity, in particular an inhibitor of ORAI2 or ORAI3 (also designated as CRACM2 or CRACM3), and optionally a pharmaceutically acceptable carrier, excipient and/or diluent. Furthermore, the present invention relates to an inhibitor of STIM2 or an inhibitor of STIM2-regulated plasma membrane calcium channel activity for use in the treatment and/or prevention of a neurodegenerative disorder associated with pathologically increased cytosolic calcium concentrations. The present invention also relates to a method of treating and/or preventing a neurodegenerative disorder associated with pathologically increased cytosolic calcium concentrations comprising administering a pharmaceutically effective amount of an inhibitor of STIM2 or of an inhibitor of STIM2-regulated plasma membrane calcium channel activity to a subject in need thereof. The present invention further relates to methods of identifying a compound suitable as a lead compound and/or as a medicament for the treatment and/or prevention of a neurodegenerative disorder associated with pathologically increased cytosolic calcium concentrations.

The solution to this technical problem is achieved by providing the embodiments characterised in the claims.

### BRIEF DESCRIPTIONS OF THE DRAWINGS

FIGS. 1a-1f represents data supporting: STIM2 as the main STIM isoform in ^{-/-} mice.

FIGS. 2a-2d represents data supporting that STIM2 regulates Ca²⁺ homeostasis in cortical neurons.

FIGS. 3a-3d represent that lack of STIM2 is neuroprotective under ischemic conditions in vitro and ex vivo.

FIGS. 4a-4c represent that Stim2^{-/-} mice are protected from neuronal damage after cerebral ischemia.

FIGS. 5a-5b represent characterization of Stim2^{-/-} mice.

FIGS. 6a-6h represent data supporting normal brain structure in Stim2^{-/-} mice.

FIGS. 7a-7b represent cognitive defects of Stim2^{-/-} mice

FIG. 8: represents sustained neuroprotection after tMCAO in Stim2^{-/-} mice.

### DETAILED DESCRIPTION

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Accordingly, the present invention relates to a pharmaceutical composition comprising an inhibitor of stromal interaction molecule 2 (STIM2) or an inhibitor of STIM2-regulated plasma membrane calcium channel activity and optionally a pharmaceutically acceptable carrier, excipient and/or diluent.

The term “pharmaceutical composition” in accordance with the present invention relates to a composition for administration to a patient, preferably a human patient. The pharmaceutical composition of the invention comprises at least one, such as at least two, e.g. at least three, in further embodiments at least four such as at last five of the above mentioned inhibitors. The invention also envisages mixtures of inhibitors of STIM2 and inhibitors of STIM2-regulated plasma membrane calcium channel activity. In cases where more than one inhibitor is comprised in the composition it is understood that none of these inhibitors has an inhibitory effect on the other inhibitors also comprised in the composition.

The composition may be in solid, liquid or gaseous form and may be, inter alia, in a form of (a) powder(s), (a) tablet(s), (a) solution(s) or (an) aerosol(s).

It is preferred that said pharmaceutical composition comprises a pharmaceutically acceptable carrier, excipient and/or diluent. Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. It is particularly preferred that said administration is carried out by injection and/or delivery, e.g., to a site in the bloodstream such as a brain or coronary artery or directly into the respective tissue. The compositions of the invention may also be administered directly to the target site, e.g., by biolistic delivery to an external or internal target site, like the brain. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 0.01 μg to 10 mg units per kilogram of body weight per minute. The continuous infusion regimen may be completed with a loading dose in the dose range of 1 ng and 10 mg/kg body weight.

Progress can be monitored by periodic assessment. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. It is particularly preferred that said pharmaceutical composition comprises further agents known in the art to antagonize neurodegeneration. Since the pharmaceutical preparation of the present invention relies on the above mentioned inhibitors, it is preferred that those mentioned further agents are only used as a supplement, i.e. at a reduced dose as compared to the recommended dose when used as the only drug, so as to e.g. reduce side effects conferred by the further agents. Conventional excipients include binding agents, fillers, lubricants and wetting agents.

The term “STIM2” relates to the “stromal interaction molecule 2” and both terms are used interchangeably herein. STIM2 is a member of the recently discovered family of endoplasmic/sarcoplasmic reticulum (ER/SR)-resident calcium-sensing molecules that regulate CCE. STIM1, another family member, has been shown to be an essential component of CCE in different cell types, including lymphocytes (Liou, 2005; Zhang, 2005; Oh-Hora, 2008), platelets (Varga-Szabo, 2008) and (excitable) skeletal muscle cells (Stiber, 2008), where it activates ORAI1 (Feske, 2006) (also termed CRACM1 (Vig, 2006)) and possibly other SOC channels. STIM2, on the other hand, has been proposed to regulate basal cytosolic calcium concentrations and store calcium concentrations (Brandman, 2007) with only a minor contribution to CCE in some cell types (Oh-Hora, 2008). The mRNA sequence of human STIM2 can be found e.g. under the NCBI accession number NM_{—}020860.2 (NCBI mRNA Reference Sequence: NM_{—}020860.2, STIM2 stromal interaction molecule 2 [Homo sapiens]; SEQ ID NO: 1 as the cDNA for STIM2).

The term “calcium” is used interchangeably with “Ca2+” or “Ca²⁺” herein. The term “[Ca²⁺],” as used throughout the present invention refers to the intracellular Ca²⁺ concentration.

The term “inhibitor” in accordance with the present invention refers to an inhibitor that reduces the biological function of a particular target protein. An inhibitor may perform any one or more of the following effects in order to reduce the biological function of the protein to be inhibited: (i) the transcription of the gene encoding the protein to be inhibited is lowered, i.e. the level of mRNA is lowered, (ii) the translation of the mRNA encoding the protein to be inhibited is lowered, (iii) the protein performs its biochemical function with lowered efficiency in the presence of the inhibitor, and (iv) the protein performs its cellular function with lowered efficiency in the presence of the inhibitor.

The inhibitor, in accordance with the present invention, may in certain embodiments be provided as a proteinaceous compound or as a nucleic acid molecule encoding the inhibitor. For example, the nucleic acid molecule encoding the inhibitor may be incorporated into an expression vector comprising regulatory elements, such as for example specific promoters, and thus can be delivered into a cell. Method for targeted transfection of cells and suitable vectors are known in the art, see for example Sambrook and Russel (“Molecular Cloning, A Laboratory Manual”, Cold Spring Harbor Laboratory, N.Y. (2001)). Incorporation of the nucleic acid molecule encoding the inhibitor into an expression vector allows to permanently elevate the level of the encoded inhibitor in any cell or a subset of selected cells of the recipient. Thus, a tissue- and/or time-dependent expression of the inhibitor can be achieved, for example restricted to neuronal cells. Thus, in a preferred embodiment, the inhibitor is a neuron-specific inhibitor.

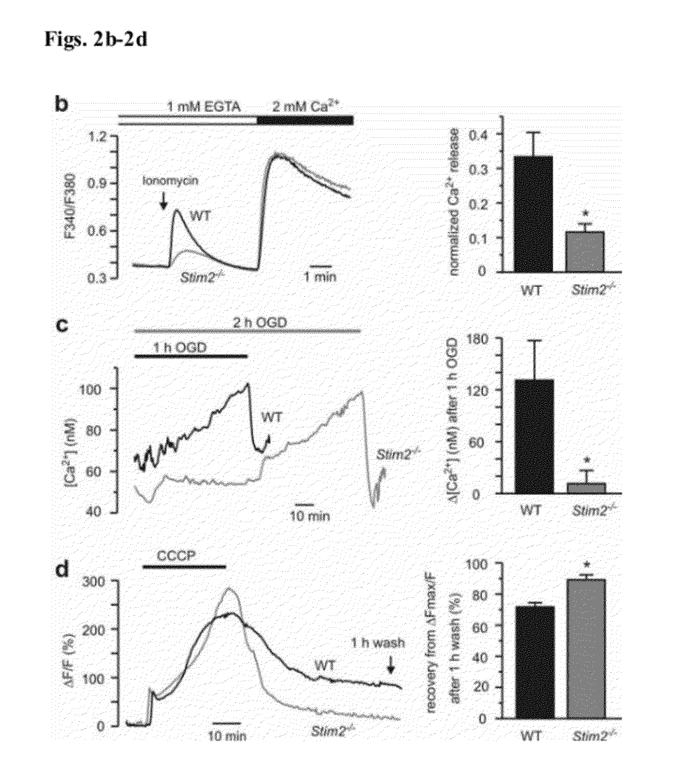

The term “inhibitor of STIM2” in accordance with the present invention refers to an inhibitor that reduces the biological function of STIM2. Biological function denotes in particular any known biological function of STIM2 including functions elucidated in accordance with the present invention. Examples of said biological function are the induction of CCE and the regulation of the basic cytosolic calcium content as well as the binding capacity of STIM2 to its downstream binding partner/s regulating the opening of the plasma membrane Ca²⁺ channel including SOC channel candidates mentioned herein such as transient receptor potential channels (TRPCs) or members of the ORAI family of channels, in particular ORAI2 and/or ORAI3, the contribution to ischemia-induced calcium entry and resulting calcium overload in neurons and neuronal cell damage or neuronal cell death. All these functions can be tested for either using any of a variety of standard methods known in the art, such as for example calcium measurements as described in FIG. 2a and example 4 or on the basis of the teachings of the examples provided below, optionally in conjunction with the teachings of the documents cited therein.

In a preferred embodiment, the inhibitor reduces the biological function of STIM2 by at least 50%, preferably by at least 75%, more preferred by at least 90% and even more preferred by at least 95% such as at least 98% or even by 100%. The term reduction by at least, for example 75%, refers to a decreased biological function such that STIM2 looses 75% of its function and, consequently, has only 25% activity remaining as compared to STIM2 that is not inhibited.

The function of any of the inhibitors referred to in the present invention may be identified and/or verified by using high throughput screening assays (HTS). High-throughput assays, independently of being biochemical, cellular or other assays, generally may be performed in wells of microtiter plates, wherein each plate may contain, for example 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to the observed biological activity.

The determination of binding of potential inhibitors can be effected in, for example, any binding assay, preferably biophysical binding assay, which may be used to identify binding test molecules prior to performing the functional/activity assay with the inhibitor. Suitable biophysical binding assays are known in the art and comprise fluorescence polarization (FP) assay, fluorescence resonance energy transfer (FRET) assay and surface plasmon resonance (SPR) assay.

In cases where the inhibitor acts by decreasing the expression level of the target protein, the determination of the expression level of the protein can, for example, be carried out on the nucleic acid level or on the amino acid level.

Methods for determining the expression of a protein on the nucleic acid level include, but are not limited to, northern blotting, PCR, RT-PCR or real RT-PCR. PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed, for example, in a 50 μl reaction mixture containing 5 μl of 10×PCR buffer with 1.5 mM MgCl₂, 200 μM of each deoxynucleoside triphosphate, 0.5 μl of each primer (10 μM), about 10 to 100 ng of template DNA and 1 to 2.5 units of Taq Polymerase. The primers for the amplification may be labeled or be unlabeled. DNA amplification can be performed, e.g., with a model 2400 thermal cycler (Applied Biosystems, Foster City, Calif.): 2 min at 94° C., followed by 30 to 40 cycles consisting of annealing (e.g. 30 s at 50° C.), extension (e.g. 1 min at 72° C., depending on the length of DNA template and the enzyme used), denaturing (e.g. 10 s at 94° C.) and a final annealing step at 55° C. for 1 min as well as a final extension step at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. However, it is well known in the art how to optimize PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746, 121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT Reaction can be performed, for example, in a 20 μl reaction mix containing: 4 μl of 5×AMV-RT buffer, 2 μl of Oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70 C.° for 15 minutes to allow for reverse transcription. The reaction temperature is then raised to 95 C.° for 1 minute to denature the RNA-cDNA duplex. Next, the reaction temperature undergoes two cycles of 95° C. for 15 seconds and 60 C.° for 20 seconds followed by 38 cycles of 90 C.° for 15 seconds and 60 C.° for 20 seconds. Finally, the reaction temperature is held at 60 C.° for 4 minutes for the final extension step, cooled to 15 C.°, and held at that temperature until further processing of the amplified sample. Any of the above mentioned reaction conditions may be scaled up according to the needs of the particular case. The resulting products are loaded onto an agarose gel and band intensities are compared after staining the nucleic acid molecules with an intercalating dye such as ethidiumbromide or SybrGreen. A lower band intensity of the sample treated with the inhibitor as compared to a non-treated sample indicates that the inhibitor successfully inhibits the protein.

Real-time PCR employs a specific probe, in the art also referred to as TaqMan probe, which has a reporter dye covalently attached at the 5′ end and a quencher at the 3′ end. After the TaqMan probe has been hybridized in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor, which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in Real time RT-PCR experiments is a DNA intercalating dye such as SybrGreen for monitoring the de novo synthesis of double stranded DNA molecules.

Methods for the determination of the expression of a protein on the amino acid level include but are not limited to western blotting or polyacrylamide gel electrophoresis in conjunction with protein staining techniques such as Coomassie Brilliant blue or silver-staining. The total protein is loaded onto a polyacrylamide gel and electrophoresed. Afterwards, the separated proteins are transferred onto a membrane, e.g. a polyvinyldifluoride (PVDF) membrane, by applying an electrical current. The proteins on the membrane are exposed to an antibody specifically recognizing the protein of interest. After washing, a second antibody specifically recognizing the first antibody and carrying a readout system such as a fluorescent dye is applied. The amount of the protein of interest is determined by comparing the fluorescence intensity of the protein derived from the sample treated with the inhibitor and the protein derived from a non-treated sample. A lower fluorescence intensity of the protein derived from the sample treated with the inhibitor indicates a successful inhibitor of the protein. Also of use in protein quantification is the Agilent Bioanalyzer technique.

The term “inhibitor of STIM2-regulated plasma membrane calcium channel activity” as used in accordance with the present invention relates to an inhibitor that directly or indirectly reduces the activity of a STIM2-regulated plasma membrane calcium channel by at least 50%, preferably by at least 75%, more preferred by at least 90% and even more preferred by at least 95% such as at least 98% or even by 100%. Non-limiting examples of the activity of a STIM2-regulated plasma membrane calcium channel include downstream binding partner(s) of STIM2 effecting the opening of the plasma membrane Ca²⁺ channel sensitive to STIM2, e.g. but not limited to ORAI1, ORAI2, ORAI3 and TRP channels and adapter molecules. Particularly the STIM2-regulated plasma membrane calcium channel activity is selected from the group consisting of ORAI2 and ORAI3. The most preferred inhibitor of STIM2-regulated plasma membrane calcium channel activity is an inhibitor of ORAI2. Examples of the biological function of ORAI2 are the binding of ORAI2 to STIM1 or STIM2 or other regulators, its function to act as a store-operated Ca²⁺ (SOC) channel, its mediation of SOCE, and in optional conjunction therewith its requirement for ischaemia-induced calcium entry and resulting calcium overload in neurons and neuronal cell damage or neuronal cell death. All these functions can be tested for by the skilled person either on the basis of common general knowledge or on the basis of the teachings of this specification, optionally in conjunction with the teachings of the documents cited therein.

The inhibitor may act directly on the calcium channel to inhibit its activity or the inhibitor may act indirectly on a regulatory molecule that in turn regulates the activity of the STIM2-regulated plasma membrane calcium channel. A variety of methods to assess the activity of a STIM2-regulated plasma membrane calcium channel are known in the art. For example, the activity of a STIM2-regulated plasma membrane calcium channel may be determined by measuring calcium entry into a test cells as described in FIG. 2a and example 4.

In accordance with the present invention it was surprisingly found that STIM2, but not STIM1, is essential for capacitive calcium entry and ischemia-induced cytosolic Ca²⁺ accumulation in neurons. Neurons from Stim2^{-/-} mice showed significantly increased survival under hypoxic conditions compared to wild-type controls both in culture and in acute hippocampal slice preparations. In vivo, Stim2^{-/-} mice were markedly protected from neurological damage in a model of focal cerebral ischemia.

Thus, it was shown in accordance with the present invention that STIM2 regulates CCE in neurons and that its absence results in altered Ca²⁺ homeostasis in these cells. This is the first direct demonstration that CCE, which is a well established phenomenon in various cell types, is of (patho-) physiological significance in cerebral neurons. Given the essential role of the closely related STIM1 for CCE in all other cell types studied so far (Oh-Hora (2008), Varga-Szabo (2008), Stiber (2008)), the central role of STIM2 instead of STIM1 in this process is surprising. Although it is not clear why neurons use STIM2, but not STIM1 to regulate CCE, it is hypothesised, without wishing to be bound, that differences between individual cell types in overall Ca²⁺ homeostasis may provide a possible explanation. The plain refilling of depleted intracellular Ca²⁺ stores, widely accepted as the foremost action of CCE in non-excitable cells, may not be a prime function in neurons since the continuous Ca²⁺ load associated with regular neuronal activity (Helmchen (1996)) should provide a sufficient supply of Ca²⁺. Furthermore, the localized dendritic Ca²⁺ release events occurring during synaptic activity (Takechi (1998)) are rapidly compensated for by shuttling of Ca²⁺ within the large continuous ER, which extends to all parts of the neurons (Berridge (1998)). Physiological roles of neuronal CCE may cover rather unexpected functions such as spontaneous transmitter release and synaptic plasticity (Emptage (2001)). Indeed, an impairment of spatial learning in Stim2^{-/-} mice similar to that observed after blockade of N-methyl-D-aspartate (NMDA)-type ionotropic glutamate receptors (Morris (1986)) was found in accordance with the present invention. For pathophysiological conditions, the results of the present invention assign a key role to CCE in Ca²⁺-dependent cell death. Anoxia reduces sarco(endo)plasmic reticulum Ca²⁺-ATPase (SERCA) activity in neurons (Goldberg (1993), Henrich (2008)), but also appears to involve active Ca²⁺ release from the ER ionositol 1,4,5 trisphosphate (IP₃) and ryanodine receptor (RyR) channels as evidenced by protection of neurons against excitotoxic injury through blockade of IP₃ receptors or RyR (Frandsen (1991) Mattson (2000)). Together, these events lead to Ca²⁺ accumulation in the cytosol and a corresponding store depletion, the latter inducing an additional Ca²⁺ load via CCE. In turn, CCE may trigger a further Ca²⁺ influx by increasing the release of glutamate and the activation of ionotropic glutamate receptors. Both, CCE and glutamatergic Ca²⁺ entry, then rapidly push the cytosolic Ca²⁺ concentration to damaging levels. Neurons devoid of STIM2 survive ischemic conditions significantly better because they do not undergo CCE and, as an additional benefit, have a lower store content, which limits the initial Ca²⁺ release and helps to better utilize the remaining Ca²⁺ sequestration capacity during the ischemic challenge.

Thus, the present invention establishes STIM2 and its downstream binding partner(s), in particular ORAI2 and/or ORAI3, as essential mediators of neuronal CCE and shows that this pathway is of paramount importance during hypoxia-induced neuronal death. Thus, the above findings allow for the preparation of pharmaceutical compositions on the basis of inhibitors of stromal interaction molecule 2 (STIM2) or inhibitors of STIM2-regulated plasma membrane calcium channel activity. Based on these findings, novel neuroprotective agents for the treatment of ischemic stroke and other neurodegenerative disorders in which disturbances in cellular Ca²⁺ homeostasis are considered a major pathophysiological component (Wojda (2008), Mattson (2007)) can now be developed. Since ORAI2 and ORAI3 are expressed in the plasma membrane it may be an even more preferred target for pharmacological inhibition as compared to STIM2 to prevent and/or treat neurological disorders associated with pathologically increased calcium concentrations.

The present invention furthermore relates to an inhibitor of stromal interaction molecule 2 (STIM2) or an inhibitor of STIM2-regulated plasma membrane calcium channel activity for use in the treatment and/or prevention of a neurodegenerative disorder associated with pathologically increased cytosolic calcium concentrations.

The term “neurological disorder” as used herein refers to any disease resulting from a deterioration of neurons or their myelin sheath, which results in dysfunctions and disabilities. Phenotypically, neurological disorders can be divided into ataxia, where the deterioration of neurons or their myelin sheaths affects movement and dementia, where the deterioration of neurons or their myelin sheaths affects memory functions. It is particularly preferred that the neurological disorder is a neurodegenerative disorder.

In accordance with the present invention, the term “neurological disorder associated with pathologically increased cytosolic calcium concentrations” relates to neurological disorders wherein a pathologically increased cytosolic calcium concentration is either causative for the disease, or is contributing to the disease or is a result of the disease. The term “pathologically increased cytosolic calcium concentration” as used hierin refers to a cytosolic calcium concentration which is increased compared to a cytosolic calcium concentration in a non-hypoxic cell. For example, overactivation of NMDA- or AMPA-receptors (e.g. by the neurotransmitter glutamate in the context of a brain injury like brain trauma or stroke) might lead to increases in intracellular calcium concentrations that lead to neuronal cell death and, consequently, to neurodegeneration. Alternatively, pathologically increased cytosolic calcium concentrations might contribute to a neurological disorder or be the result of a neurological disorder e.g. in spinal cord injury, stroke, traumatic brain injury, epilepsy, multiple sclerosis, Alzheimer's disease, amyotrophic lateral sclerosis (ALS), Parkinson's disease and Huntington's disease.

Alternatively, the mentioned inhibitor STIM2 or the inhibitor of STIM2-regulated plasma membrane calcium channel activity may be used as a lead compound for the development of a drug for treating and/or preventing a disorder associated with pathologically increased cytosolic calcium concentrations. Those lead compounds will also allow for the development of novel, highly effective, yet safe neuroprotective agents. In the development of those drugs, the following developments are considered: (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmacokinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carboxylic acids, or (iii) esterification of hydroxyl groups to, e.g. phosphates, pyrophosphates or sulfates or hemi-succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatization of hydroxyl group to ketales, acetales, or (xiv) N-acetylation to amides, phenylcarbamates, or (xv) synthesis of Mannich bases, imines, or (xvi) transformation of ketones or aldehydes to Schiffs bases, oximes, acetales, ketales, enolesters, oxazolidines, thiazolidines or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The present invention further relates to a method of treating and/or preventing a neurological disorder caused by pathologically increased cytosolic calcium concentrations comprising administering a pharmaceutically effective amount of an inhibitor of STIM2 or of an inhibitor of STIM2-regulated plasma membrane calcium channel activity to a subject in need thereof.

In a preferred embodiment of the pharmaceutical composition or the inhibitor or the method of the invention, the inhibitor is an antibody or a fragment or derivative thereof, an aptamer, a siRNA, a shRNA, a miRNA, a ribozyme, an antisense nucleic acid molecule, modified versions of these inhibitors or a small molecule.

The term “antibody” as used in accordance with the present invention comprises, for example, polyclonal or monoclonal antibodies. Furthermore, also derivatives or fragments thereof, which still retain the binding specificity, are comprised in the term “antibody”. Antibody fragments or derivatives comprise, inter alia, Fab or Fab′ fragments as well as Fd, F(ab′)₂, Fv or scFv fragments; see, for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanized (human antibody with the exception of non-human CDRs) antibodies.

Various techniques for the production of antibodies are well known in the art and described, e.g. in Harlow and Lane (1988) and (1999), loc. cit. Thus, the antibodies can be produced by peptidomimetics. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to an epitope of STIM2 or an epitope of a STIM2-regulated plasma membrane calcium channel (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold.

Nucleic acid aptamers are nucleic acid species that, as a rule, have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers usually are peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

The term “peptide” as used herein describes a group of molecules consisting of up to 30 amino acids, whereas the term “protein” as used herein describes a group of molecules consisting of more than 30 amino acids. Peptides and proteins may further form dimers, trimers and higher oligomers, i.e. consisting of more than one molecule which may be identical or non-identical. The corresponding higher order structures are, consequently, termed homo- or heterodimers, homo- or heterotrimers etc. The terms “peptide” and “protein” (wherein “protein” is interchangeably used with “polypeptide”) also refer to naturally modified peptides/proteins wherein the modification is effected e.g. by glycosylation, acetylation, phosphorylation and the like. Such modifications are well-known in the art.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene of which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1-5 nucleotides, more preferably from 1-3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules which, as endogenous RNA molecules, regulate gene expression. Binding to a complementary mRNA transcript triggers the degradation of said mRNA transcript through a process similar to RNA interference. Accordingly, miRNA may be employed as an inhibitor of STIM2 or an inhibitor of STIM2-regulated plasma membrane calcium channel activity, in particular ORAI2 and/or ORAI3.

A ribozyme (from ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in the last 10 years. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

A recent development, also useful in accordance with the present invention, is the combination of an aptamer recognizing a small compound with a hammerhead ribozyme. The conformational change induced in the aptamer upon binding the target molecule is supposed to regulate the catalytic function of the ribozyme.

The term “antisense nucleic acid molecule” is known in the art and refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

The term “modified versions of these inhibitors” in accordance with the present invention refers to versions of the inhibitors that are modified to achieve i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (k) conversion of alkyl substituents to cyclic analogues, or (l) derivatization of hydroxyl groups to ketales, acetales, or (m) N-acetylation to amides, phenylcarbamates, or (n) synthesis of Mannich bases, imines, or (o) transformation of ketones or aldehydes to Schiffs bases, oximes, acetales, ketales, enolesters, oxazolidines, thiazolidines; or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

A “small molecule” according to the present invention may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu (atomic mass units), or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vitro assays such as in vitro high-throughput screening (HTS) assays.

In another preferred embodiment of the pharmaceutical composition or the inhibitor or the method of the invention, the STIM2-regulated plasma membrane calcium channel is selected from the group consisting of ORAI1, ORAI2, ORAI3 and TRP channels. In the more preferred embodiment of the pharmaceutical composition or the inhibitor or the method of the invention, the STIM2-regulated plasma membrane calcium channel is ORAI2 and/or ORAI3. In the most preferred embodiment the STIM2-regulated plasma membrane calcium channel is ORAI2.

The terms “ORAI1”, “ORAI2” and “ORAI3” as used herein refer to members of the ORAI protein family (also called CRACM). The proteins of this family are plasma membrane proteins that form four transmembrane segments. The NCBI accession numbers for the proteins of the ORAI-family-are: NM_{—}032790.3 for ORAI1 (ORAI calcium release-activated calcium modulator 1); NM_{—}001126340.1 for ORAI2 (ORAI calcium release-activated calcium modulator 2, transcript variant 1), NM_{—}032831.2 for ORAI2 (ORAI calcium release-activated calcium modulator 2, transcript variant 2) and NM_{—}152288.2 for ORAI3 (ORAI calcium release-activated calcium modulator 3).

The term “TRP channels” as used in accordance with the present invention refers to a family of transient receptor potential (TRP) channel proteins which form subunits having six transmembrane domains that are assumed to assemble into tetramers to form non-selective cationic channels, which allow for the influx of calcium ions into cells. The TRP channel proteins are encoded by at least 33 channel subunit genes divided into seven sub-families comprising TRPC (canonical), TRPV (vanilloid), TRPA (ankyrin), TRPM (melastatin), TRPP (polycystin), TRPML (mucolipin) and TRPN(NOMPC—no mechanoreceptor potential C). In a more preferred embodiment, the TRP channel is selected from TRPC or TRPM channels. In a further more preferred embodiment, the TRPC channel is TRPC1.

In another preferred embodiment, the pharmaceutical composition of the invention or the inhibitor of the invention further comprises in the same or a separate container a neuroprotective and/or neuroregenerative substance and/or an antithrombotic substance.

Further the invention relates to a combined pharmaceutical composition of an inhibitor of stromal interaction molecule 2 (STIM2) or an inhibitor of STIM2-regulated plasma membrane calcium channel activity, in particular an inhibitor of ORAI2 and/or ORAI3, and a neuroprotective and/or neuroregenerative substance and/or an antithrombotic substance for the simultaneous, separate or sequential use in therapy. This combined pharmaceutical composition optionally contains a pharmaceutically active carrier, excipient and/or diluent.

In a preferred embodiment the use in therapy for this combined pharmaceutical composition is the use in treating and/or preventing a disorder associated with pathologically increased cytosolic calcium concentrations or as a lead compound for developing a drug for treating or preventing a disorder associated with pathologically increased cytosolic calcium concentrations.

The term “neuroprotective substance” in accordance with the present invention relates to substances which protect the nervous system, and in particular neuronal cells, from degeneration or cell death (apoptosis and/or necrosis), for example following a brain injury or as a result of chronic neurodegenerative diseases. The term “neuroregenerative substance” as used throughout the present invention refers to a substance that stimulates or enhances growth and regeneration of parts of the nervous system, in particular of neuronal cells.

The term “antithrombotic substance” in accordance with the present invention refers to substances capable of reducing thrombus formation, i.e. the formation of blood clots. Thrombus formation can for example be reduced by either limiting the migration or aggregation of platelets, or by limiting the ability of the platelets to clot using anticoagulants or by dissolving blood clots after they have formed using thrombolytic substances. It is particularly preferred that the antithrombotic substance is a thrombolytic substance.

In a more preferred embodiment, the neuroprotective and/or neuroregenerative substance is selected from the group consisting of a glutamate antagonist (e.g. but not limited to glutamate receptor antagonists and glutamate release inhibitors), a free-radical reducing agent (i.e. an antioxidant or a free-radical scavenger), a calcium antagonist (e.g. but not limited to a calcium channel blocker and a calcium chelator), a potassium channel activator, a GABA agonist, an opiate antagonist, a leukocyte adhesion inhibitor, an inhibitor of cytokines, a membrane stabilizer, a neutrophil modulator, a glycine antagonist, an apoptosis modulator, a neuronal guidance modulator, a neurotrophic factor and a stem cell modulator.

In accordance with the present invention, the term “glutamate antagonist” refers to a substance which antagonizes or inhibits the effects of glutamate activity either by blocking glutamate receptors, inhibition of glutamate release, enhancement of glutamate uptake or through other mechanisms. Non-limiting examples of glutamate antagonists include Aptiganel (CNS-1102, Cerestat), Gavestinel, Dextrorphan, CGS 19755 (Selfotel), Eliprodil, ACEA 1021, YM872, ZK-200775, magnesium, Sipatrigine and Fosphenyloin.

The term “free-radical reducing agent” according to the present invention refers to substances or systems, capable of slowing or preventing the oxidation of other molecules, or capturing free radicals. Non-limiting examples of free-radical reducing agentsinclude vitamine C, vitamin E, NXY-059 (Cerovive), Ebselen, Tirilazade mesylate and Edaravone.

In accordance with the present invention, the term “calcium antagonist” refers to a substance which antagonizes or inhibits the effects of calcium activity either by blocking calcium receptors, inhibition of calcium release, capturing of free calcium or through other mechanisms. Non-limiting examples of calcium antagonists include dihydropyridines (e.g. Nimodipine), Flunarizine and DP-b99.

The term “potassium channel activator” according to the present invention refers to a substance which activates potassium channels. Potassium channels are expressed in many tissues including brain, pancreatic β-cells, heart and smooth muscle. Amongst others, activation of potassium channels in vascular smooth muscle causes membrane hyperpolarization and arterial vasodilation. Nicorandil also has nitrate-like venodilatory effects. Non-limiting examples of potassium channel activators include BMS-204352 and Nicorandil.

The term “GABA agonist” as used throughout the present invention refers to a substance which stimulates or increases the action at the GABA receptor (GABA (gamma-aminobutyric acid) is the chief inhibitory neurotransmitter in the vertebrate central nervous system). Amongst others, anxiolytic, sedative and muscle relaxant effects can occur. Non-limiting examples of GABA agonists include Clomethiazole and Diazepam.

The term “opiate antagonist” as used throughout the present invention refers to a substance which antagonizes or inhibits the effects of opiate activity. Non-limiting examples of opiate antagonists include Nalmefene (Cervene, ReVex) and Naloxone.

In accordance with the present invention, the term “leukocyte adhesion inhibitor” refers to a substance, which antagonizes or inhibits the ability of leukocytes to recognize and adhere to cellular substrates—amongst others, an important function in inflammatory processes. Non-limiting examples of leukocyte adhesion inhibitors include Anti-ICAM-1 antibody (Enlimomab) and HU23F2G.

The term “inhibitor of cytokines” according to the present invention refers to a substance which antagonizes or inhibits the effects of cytokines Non-limiting examples of inhibitors of cytokines include IL-1 receptor antagonists.

The term “membrane stabilizer” as used throughout the present invention refers to a substance which stabilizes cellular membranes, thus interfering with dysfunctional processes. Non-limiting examples of membrane stabilizers include Citicoline.

The term “neutrophil modulator” as used throughout the present invention refers to a substance which modulates the function of neutrophils. The modulation of neutrophil activities constitutes an approach to regulate inflammatory responses, with the desired effect being a balance between the protective and tissue repair properties of neutrophils and their tissue destroying potential. Non-limiting examples of neutrophil modulators include neutrophil inhibitory factor.

The term “glycine antagonist” according to the present invention refers to a substance which antagonizes or inhibits the effects of glycine activity either by blocking glycine receptors or binding sites, or through other mechanisms. Non-limiting examples of glycine antagonists include GV150526 (Gavastinel).

The term “apoptosis modulator” as used throughout the present invention refers to a substance which interferes with apoptotic cell signaling either by inhibiting proteins involved in apoptotic processes or through other mechanisms. Non-limiting examples of apoptosis modulators are melatonin.

In accordance with the present invention, the term “neuronal guidance modulator” refers to a substance which modulates neuronal guidance processes either by enhancing axonal outgrowth or directing axonal outgrowth or through other mechanisms. Non-limiting examples of neuronal guidance modulators include nerve growth factor.

The term “neurotrophic factor” as used throughout the present invention refers to a substance which, amongst others, is capable of influencing neuronal survival, differentiation or growth. Non-limiting examples of neurotrophic factors are brain derived neurothrophic factor, nerve growth factor and neurotrophins.

The term “stem cell modulator” according to the present invention refers to a substance which, amongst others, is capable of influencing stem cell growth, differentiation and guidance. Non-limiting examples of stem cell modulators are stem cell factor.

In another more preferred embodiment, the antithrombotic substance is recombinant tissue plasminogen activator, but also any other thrombolytic substances.

The term “recombinant tissue plasminogen activator”, which is interchangeably used herein with the abbreviation rTPA, in accordance with the present invention is well known to the skilled person and refers to recombinantly produced tissue plasminogen activator, which is a naturally occurring fibrinolytic agent found in vascular endothelial cells. Tissue plasminogen activator is involved in the balance between thrombolysis and thrombogenesis. It exhibits significant fibrin specificity and affinity. At the site of the thrombus, the binding of tPA and plasminogen to the fibrin surface induces a conformational change facilitating the conversion of plasminogen to plasmin and dissolving the clot.

In a further more preferred embodiment, the neurological disorder associated with pathologically increased cytosolic calcium concentrations is selected from cerebral ischemia, brain stroke (i.e. ischemic stroke and hemorrhagic stroke), Alzheimer's disease, Parkinson's disease, Huntington's disease, autosomal dominant spinocerebellar ataxias, glaucoma, amyotrophic lateral sclerosis, epilepsy, schizophrenia, traumatic brain injury and HIV dementia.

The term “cerebral ischemia” as used throughout the present invention relates to a reduction of blood flow to the brain, thus leading to brain damage. Cerebral ischemia is also any situation in which the flow of blood to the brain is not a sufficient amount that will meet the metabolic demands of brain tissue. Cerebral ischemia has been connected to cerebral hypoxia, i.e. the deprivation of oxygen to brain tissue and, if prolonged, leads to an ischemic stroke.

The term “brain stroke” as used in accordance with the present invention refers to the disruption of cerebral blood flow followed by rapidly developing loss of brain function(s). Brain stroke may due to ischemia (ischemic stroke; lack of blood supply) caused by thrombosis that is the formation of blood clots inside the blood vessels, or embolism i.e. the blockade of a blood vessel by an object that has migrated from another part of the body, or hypoperfusion described above. Brains stroke may also be due to a hemorrhage (hemorrhagic stroke). A prolonged elevation of intracellular calcium is observed as a consequence of the disruption of cerebral blood flow, which results in neuronal death (Wojda, 2008).

In accordance with the present invention, “Alzheimer's disease” refers to an age-related neurodegenerative chronic dementia characterized by slow, gradual degeneration and death of neurons in the forebrain and particularly in the hippocampus. Affected brain areas of Alzheimer's disease patients show amongst others increased levels of calcium and increased activation of calcium-dependent enzymes. It has been suggested that the etiology of Alzheimer's disease is based on the interplay between calcium dyshomeostasis and neuropathological hallmarks of Alzheimer's disease such as Amyloid beta (Aβ) and hyperphosphorylated tau protein (Wojda, 2008).

“Parkinson's disease” in accordance with the present invention is a progressive neurodegenerative disease and the most common motor system disorder strongly associated with ageing. Symptoms such as bradykinesia, rigidity, tremor and other motor symptoms are attributed to the selective degeneration and loss of dopaminergic neurons in the substantia nigra pars compacta. Calcium dyshomeostasis, when exacerbated by environmental insults, such as heavy metals, pesticides, neurotoxins or inflammation or due to genetic predispositions, has been associated with neurodegeneration in Parkinson's disease (Wojda, 2008).

“Huntington's disease”, according to the present invention, refers to an inherited autosomal dominant neurodegenerative disease characterized by motor and psychiatric symptoms such as chorea and gradual dementia connected to a selective and progressive loss of medium spiny neurons in the striatum. Huntington's disease is caused by the abnormal protein huntingtin (Ht), which contains polyglutamine expansions in the N-terminal region. Some toxic functions assigned to mutant Ht, such as disruption of mitochondrial calcium homeostasis or malfunction of the ER calcium store due to sensitization of the IP3R to its activation by IP3, convert into calcium dyshomeostasis (Wojda, 2008).

In accordance with the present invention, “autosomal dominant spinocerebellar ataxias” refer to a complex group of neurodegenerative disorders characterized by progressive cerebellar ataxia of gait and limbs variably associated with ophtalmoplegia, pyramidal and extrapyramidal signs, dementia, pigmentary retinopathy and peripheral neuropathy. Neuronal calcium signaling disturbances are believed to be involved in the neurodegeneration of spinocerebellar ataxias (Wojda, 2008).

“Glaucoma” according to the present invention relates to a group of neurodegenerative diseases resulting in blindness. Glaucoma is characterized by structural damage to the optic nerve and slow progressive death of retinal ganglion cells (Wojda, 2008).

“Amyotrophic lateral sclerosis”, in accordance with the present invention, refers to a multifactoral neurodegenerative disease, characterized by progressive and highly selective loss of cortical, spinal and brainstem motor neurons and is accompanied by the progressive loss of muscle force and breathing capacity, swallowing difficulties, and limb spasticity. Disruption of intracellular calcium homeostasis, including glutamate excitotoxicity, calcium-dependent formation of protein aggregates and calcium-evoked mitochondrial dysfunction, are thought to play a key role in the mechanisms leading to this selective degradation of motor neurons (Wojda, 2008).

In accordance with the present invention, “epilepsy” refers to a chronic neurological condition, characterized by an uncontrolled, excessive electric discharge by the neurons resulting in unprovoked seizures. Injury-induced alterations in calcium homeostasis have been suggested to play a role in the development and maintenance of acquired epilepsy (Wojda, 2008).

“Schizophrenia” according to the present invention refers to a psychiatric disorder characterized by abnormalities in the perception or expression of reality. It most commonly manifests as auditory hallucinations, paranoid or bizarre delusions, or disorganized speech and thinking with significant social or occupational dysfunction. Altered intracellular calcium signaling is considered to potentially play a crucial role for the molecular mechanisms leading to schizophrenia (Wojda, 2008).

“Traumatic brain injury”, in accordance with the present invention, refers to brain damage after head injury, which may be divided into contact or acceleration/deceleration types of injury. Contact injury usually results in focal brain damage, whereas acceleration/deceleration injuries lead to diffuse brain damage characterized by widespread axons damage, ischemic brain injury and diffuse brain swelling. Dramatic increases in the extracellular level of glutamate after traumatic brain injury results in over-stimulation of excitatory amino acids receptors and excessive calcium influx into neurons. Furthermore, traumatic deformation of axons induces abnormal sodium influx through mechanically sensitive sodium channels, which subsequently triggers an increase in intra-axonal calcium. This severe disturbance of the calcium homeostasis in neurons eventually results in cell injury and death (Wojda, 2008).

“HIV dementia” in accordance with the present invention is a dementia caused by the effect of a human immunodeficiency virus type-1 (HIV-1) infection. The disease often results in loss of cortical neurons and retinal ganglion cells (RGC), accompanied by a loss in the complexity of dendritic arborization. The viral gp120 protein is thought to contribute to HIV dementia via its ability to modify NMDA-receptor kinetics, which results in neuronal calcium overload and cellular destruction or death by calcium-triggered mechanisms. The viral Tat protein, via pertussis toxinsensitive phospholipase C activity, induces calcium release from IP3-sensitive intracellular stores, which is followed by glutamate receptor-mediated calcium influx and neuronal cell death (Wojda, 2008).

All of the diseases described herein are well known to the skilled person and are defined in accordance with the prior art and the common general knowledge of the skilled person.

The present invention also relates to a method of identifying a compound suitable as a lead compound and/or as a medicament for the treatment and/or prevention of a neurodegenerative disorder associated with pathologically increased cytosolic calcium concentrations, comprising the steps of (a) determining the level of STIM2 protein or Stim2 transcript in a cell; (b) contacting said cell or a cell of the same cell population with a test compound; (c) determining the level of STIM2 protein or Stim2 transcript in said cell after contacting with the test compound; and (d) comparing the level of STIM2 protein or Stim2 transcript determined in step (c) with the STIM2 protein or Stim2 transcript level determined in step (a), wherein a decrease of STIM2 protein or Stim2 transcript level in step (c) as compared to step (a) indicates that the test compound is a compound suitable as a lead compound and/or as a medicament for the treatment and/or prevention of a neurodegenerative disorder associated with pathologically increased cytosolic calcium concentrations.

This embodiment relates to a cellular screen, wherein inhibitors may be identified which exerts their inhibitory activity by interfering with the expression of STIM2, either by affecting the stability of STIM2 protein or transcript (mRNA) or by interfering with the transcription or translation of STIM2.

A “compound” in accordance with the present invention can be any of the inhibitors defined above, i.e. an antibody or a fragment or derivative thereof, an aptamer, a siRNA, a shRNA, a miRNA, a ribozyme, an antisense nucleic acid molecule, modified versions of these inhibitors or a small molecule. Test compounds further include but are not limited to, for example, peptides such as soluble peptides, including Ig-tailed fusion peptides and members of random peptide libraries (see, e.g., Lam et al. (1991) Nature 354: 82-84; Houghten et al. (1991) Nature 354: 84-86) and combinatorial chemistry-derived molecular libraries made of D- and/or L-configuration amino acids or phosphopeptides (e.g., members of random and partially degenerate, directed phosphopeptide libraries, see, e.g., Songyang et al. (1993) Cell 72: 767-778).

The term “said cell or a cell of the same cell population” as used herein refers either to the cell used in step (a) or to a cell being of the same origin as the cell of step (a) and that is identical in its characteristics to the cell of (a). Furthermore, this term also encompasses cell populations, such as for example homogenous cell populations consisting of cells having identical characteristics, and, thus, is not restricted to single cell analyses.

As described hereinabove, STIM2 is an essential mediator of neuronal CCE. Therefore, the use of STIM2 as a target for the discovery of compounds that are suitable for the treatment and/or prevention of a neurodegenerative disorder associated with pathologically increased cytosolic calcium concentrations is also encompassed by the present invention. It is envisaged that a decrease of expression levels of STIM2 conferred by a compound as described above may contribute to neuroprotection from pathologically increased cytosolic calcium concentrations and may ameliorate conditions associated therewith, as described above. Accordingly, measurement of the STIM2 protein or Stim2 transcript level may be used to determine the readout of the above-described assay.

For example, the above-mentioned cell may exhibit a detectable level of STIM2 protein or Stim2 transcript before contacting with the test compound and the level of STIM2 protein or Stim2 transcript may be lower or undetectable after contacting the cell with the test compound, indicating a compound suitable for the treatment and/or prevention of a neurodegenerative disorder associated with pathologically increased cytosolic calcium concentrations or as a lead compound for the development of a compound for this treatment. Preferably, the level of STIM2 protein or Stim2 transcript after contacting the cell with the test compound is reduced by, for example, at least 10, 20, 30, 40 or 50% as compared to the level of STIM2 protein or Stim2 transcript before contacting the cell with the test compound. More preferably, the level of STIM2 protein or Stim2 transcript after contacting the cell with the test compound is reduced by, for example, at least 60, 70, 80, 90 or 95% as compared to the level of STIM2 protein or Stim2 transcript before contacting the cell with the test compound. Most preferably, the level of STIM2 protein or Stim2 transcript after contacting the cell with the test compound is reduced by 100% as compared to the level of STIM2 protein or Stim2 transcript before contacting the cell with the test compound. The term “the level of STIM2 protein or Stim2 transcript is reduced by . . . %” refers to a relative decrease compared to the level of STIM2 protein or Stim2 transcript before contacting the cell with the test compound. For example, a reduction of at least 40% means that after contacting the cell with the test compound the remaining level of STIM2 protein or Stim2 transcript is only 60% or less as compared to the level of STIM2 protein or Stim2 transcript before contacting the cell with the test compound. A reduction by 100% means that no detectable level of STIM2 protein or Stim2 transcript remains after contacting the cell with the test compound.

Measurements of protein levels as well as of transcript level can be accomplished in several ways, as described above.

In a preferred embodiment, the method is carried out in vitro. In vitro methods offer the possibility of establishing high-throughput assays, as described above.

The identified so-called lead compounds may be optimized to arrive at a compound which may be used in a pharmaceutical composition. Methods for the optimization of the pharmacological properties of compounds identified in screens, the lead compounds, are known in the art and comprise, for example, the methods described above for the modified versions of the preferred inhibitors of the invention.

The present invention further relates to a method of identifying a compound suitable as a lead compound and/or as a medicament for the treatment and/or prevention of a neurodegenerative disorder associated with pathologically increased cytosolic calcium concentrations, comprising the steps of: (a) emptying the intracellular calcium stores of a cell containing STIM2 protein in the absence of extracellular calcium and determining the increase in intracellular calcium concentration upon addition of extracellular calcium; (b) contacting said cell or a cell of the same cell population containing STIM2 protein with a test compound; (c) after contacting with the test compound, emptying the intracellular calcium stores of the cell of (b) in the absence of extracellular calcium and determining the increase in intracellular calcium concentration upon addition of extracellular calcium in said cell; and (d) comparing the increase in intracellular calcium concentration determined in step (c) with the increase in intracellular calcium concentration determined in step (a), wherein no increase in intracellular calcium concentration or a smaller increase in intracellular calcium concentration in step (c) as compared to step (a) indicates that the test compound is a compound suitable as a lead compound and/or as a medicament for the treatment and/or prevention of a neurodegenerative disorder associated with pathologically increased cytosolic calcium concentrations.

This embodiment relates to a cellular screen, wherein inhibitors may be identified which exert their inhibitory activity by physically interacting with STIM2, or alternatively (or additionally) by functionally interacting with STIM2, i.e., by interfering with the pathway(s) present in the cells employed in the cellular assay, as well as by physically and/or functionally interacting with the STIM2-regulated plasma membrane calcium channel activity, i.e. by interfering with the downstream binding partner(s) of STIM2. As a result, the biological activity of STIM2 or the STIM2-regulated plasma membrane calcium channel is altered either directly or indirectly.

This method includes as a first step the emptying of the intracellular calcium stores of a cell containing STIM2 protein. Suitable compounds for calcium release from the intracellular stores are well known in the art and include, without being limiting, cyclopiazonic acid (CPA) and thapsigargin. The stores are emptied in the absence of extracellular calcium, which may for example be achieved by performing this step in the presence of the calcium chelator EGTA or EDTA. The capacity of the cell to perform STIM2-mediated CCE is then determined by adding extracellular calcium and, where necessary, the removal of calcium chelating agents such as EGTA. In cells capable of STIM2-mediated CCE, a corresponding increase in the intracellular calcium concentration will be observed. An example for how to determine the increase of intracellular calcium as described above is given in Example 4 and in particular in FIG. 2(a).

In the next step of the method, the cell or a cell of the same cell population containing STIM2 protein is contacted with a test compound and, in a third step, the ability of said cell to perform STIM2-mediated CCE after contacting with the test compound is determined as described above. Again, STIM2-mediated CCE is determined based on the increase in the intracellular calcium concentration observed. The thus observed increase in the amount of intracellular calcium is compared between the first step carried out in the absence of the test compound and the third step, carried out after contacting with the test compound. If after contacting with the test compound no increase in intracellular calcium at all is observed, then the test compound has successfully inhibited CCE completely, i.e. to 100%, and is thus suitable as a lead compound and/or as a medicament for the treatment and/or prevention of a neurodegenerative disorder associated with pathologically increased cytosolic calcium concentrations. The test compound is also suitable as a lead compound and/or as a medicament for the treatment and/or prevention of said diseases if a smaller increase in intracellular calcium concentration is observed after contacting with the test compound as compared to the increase observed in the absence of the test compound. This smaller increase can be in accordance with the present invention lower than 70%, preferably lower than 50%, more preferred lower than 40% and even more preferred lower than 30% such as at least lower than 20% as compared to the increase observed in the absence of the test compound.

In a preferred embodiment of the method of the invention, the cell comprising the STIM2 protein is a neuronal cell.

The term “neuronal cell” in accordance with the present invention refers to any one of the cells of the nervous system, i.e. the brain, the spinal cord and the peripheral nerves, that process and transmit information by electrochemical signaling. Preferably, said neuronal cell is a primary cortical neuron or alternatively a hippocampal neuron.

### EXAMPLES

The examples illustrate the invention.

### Example 1

### Materials and Methods

Mice. Experiments were conducted in accordance with the regulations of the local authorities (Regierung von Unterfranken) and were approved by the institutional review boards of all participating institutions. Stim2^{-/-} knockout mice were generated by deletion of most parts of exons four to seven of the Stim2 gene using standard molecular techniques. Briefly, two genomic DNA clones encoding the mouse Stim2 gene (RPCI22-446E8; RPCI22-394122) where isolated from a 129Sv BAC library (Chori, USA), using a probe specific for mouse Stim2 exon 4 previously isolated by PCR. The targeting construct was designed to delete most of the exons four to seven. Stim2 was targeted by homologous recombination in R1 embryonic stem (ES) cells derived from 129Sv mouse strain. Targeted stem cell clones were screened by Southern blot using a gene specific external probe. Stim2^{+/-} ES cells were injected in C57B1/6 blastocysts to generate chimeric mice. Chimeric mice were crossed back with C57B1/6 (Harlan Laboratories) and subsequent progenies were intercrossed to obtain Stim2^{-/-} mice. Genotypes were determined by PCR analysis using the following primer pairs:

Stim2 wild-type 5′:

SEQ ID NO: 5

CCCATATGTAGATGTGTTCAG;
Stim2 wild-type 3′:

SEQ ID NO: 6

GAGTGTTGTTC-CCTTCACAT.;
Stim2 knock-out 5′:

SEQ ID NO: 7

TTATCGATGAGCGTGGTGGTTATGC;
Stim2 knock-out 3′:

SEQ ID NO: 8

GCGCGTACATCGGGCAAATAATATC.

For the generation of bone marrow chimeras, 5-6 weeks old Stim2^{+/+} and Stim2^{-/-} female mice were irradiated with a single dose of 10 Gy, and bone marrow cells from 6 weeks old Stim2^{+/+} or Stim2^{-/-} mice from the same litter were injected intravenously into the irradiated mice (4×10⁶ cells/mouse).

Stim1^{-/-} and Orai1^{-/-} mice were generated as described before (Varga-Szabo (2008), Braun (2008)).

The NCBI accession numbers for the mRNA sequences of the mice proteins STIM2, STIM1 and ORAI1 are: NM_{—}001081103.1 for Mus musculus stromal interaction molecule 2 (STIM2; SEQ ID NO: 2 as the cDNA for STIM2); NM_{—}009287.4 for Mus musculus stromal interaction molecule 1 (STIM1; SEQ ID NO: 3 as the cDNA for STIM1); NM_{—}175423.3 for Mus musculus ORAI calcium release-activated calcium modulator 1 (Orai1; SEQ ID NO: 4 as the cDNA for ORAI1).

Western Blots. Western blotting was performed with standard protocols using total protein lysates from different mouse organs extracted with RIPA buffer. The following primary antibodies were used for western blotting: rabbit anti-STIM2-CT (1:400, ProSci), rabbit anti-STIM1 (1:500, Cell Signaling), rat anti-α-tubulin (1:1000, Chemicon international). All HRP-conjugates antibodies were purchased from Jackson ImmunoResearch (Suffolk, UK) or Dianova (Hamburg, Germany). Bound antibodies were detected with enhanced chemiluminescent Western Lightning™ Plus-ECL (Perkin Elmer).

RT-PCR analysis. Mouse mRNA from 50 single neurons isolated by laser capture micro-dissection was extracted and reverse-transcribed. Stim1 and Stim2 mRNA was detected by amplification of the 3′ region (most heterogeneous region among STIMs) by RT-PCR using specific primers, as follows:

| | | |
|---|---|---|
| Stim1RT 5′: | CTTGGCCTGGGATCTCAGAG; | SEQ ID NO: 9 |
| Stim1RT 3′: | TCAGCCATTGCCTTCTTGCC; | SEQ ID NO: 10 |
| Stim2RT 5′: | GCAGGATCTTTAGCCAGAAG; | SEQ ID NO: 11 |
| Stim2RT 3′: | ACATCTGCTGTCACGGGTGA. | SEQ ID NO: 12 |

Orai primers were used as previously described (Braun (2008)).

Histology. Paraffin (Histolab Products AB) paraformaldehyde-fixed organs were cut into 5-μm thick sections and mounted. After removal of paraffin, tissues were stained with hematoxylin and eosin (Sigma-Aldrich) or with Nissl staining method following standard protocols.

Immunocytochemistry. Cultured hippocampal cell cultures were fixed with 4% PFA (Merck, Germany), washed 3 times with 10 mM PBS and incubated for 60 min at 4° C. in 10 mM PBS containing 5% goat serum (GS; PAA Laboratories, Austria) and 0.3% Triton X100 (Sigma, Germany). Primary antibodies (mouse MAP2a/b 1:200, abcam, United Kingdom; rabbit Cleaved Caspase-3 1:150, Cell Signaling, MA) were diluted in 10 mM PBS and incubated for 12 hours at 4° C. After washing steps with 10 mM PBS, incubation with secondary antibodies (Alexa 488 labeled goat anti-mouse 1:100, BD Bioscience, Cy-3 labeled goat anti-rabbit 1:300, Dianova, Hamburg) was carried out in the same manner. Staining with 0.5 μg/ml DAPI (Merck, Germany) was performed for 7 min. Finally, cultures were washed and subsequently covered with DABCO (Merck, Darmstadt). Pictures were collected by immunofluorescence microscopy (Axiophot; Zeiss, Jena).

In one set of experiments, cultured neuronal cells as well as isolated murine CD4′ T cells from wild-type mice were placed on coverslips coated with poly-L-lysine (Sigma, Deisenhofen, Germany), fixed with 4% PFA and stained with anti-STIM1 (Cell Signaling, MA) or anti-STIM2 (Cell Signaling, MA) antibodies followed by Cy-3 labeled goat anti-rabbit IgG, Dianova, Hamburg). Cell nuclei were stained with DAPI (Merck, Darmstadt, Germany).

Neuronal cell cultures. Neuronal cultures were obtained from Stim1^{-/-}, Stim2^{-/-} or control mice (E18) according to previously described preparation protocols (Meuth (2008)). In brief, pregnant mice were killed by cervical dislocation and embryos were removed and transferred into warm Hank's buffered salt solution (HBSS). After preparation of hippocampi, the tissue was collected in a tube containing 5 ml of 0.25% trypsin in HBSS. After five minutes of incubation at 37° C., the tissue was washed twice with HBSS. Thereafter, the tissue was dissociated in 1 ml of neuronal medium by triturating with fire polished pasteur pipettes of decreasing tip diameter. Neurons were diluted in neuronal medium (10% 10× modified earl's medium (MEM); 0.2205% sodium bicarbonate; 1 mM sodium pyruvate; 2 mM L-glutamine; 2% B27 supplement (all Gibco, Germany); 3.8 mM glucose; 1% penicillin/streptomycin (Biochrom AG, Germany), and plated in a density of 60,000 cells/cm² on poly-D-lysine coated cover slips in 4-well plates (Nunc, Denmark). Prior to experiments all cell cultures were incubated at 37.0° C. and 5% CO₂ and held in culture for up to 5-7 days. To induce ischemic conditions we changed the pH to 6.5, lowered glucose concentrations and restricted O₂ as described for the slice preparations.

For calcium measurements, primary neuronal cultures were obtained from Stim1^{-/-}, Stim2^{-/-}, Orai1^{-/-} or control mice (E18-P0) as described above except for the following: Tissue was collected from whole cortices and cells were cultured in Neurobasal-A medium containing 2% B27 supplement, 1% GlutaMAX-1 and 1% penicillin/streptomycin (all Gibco, Germany). Cells were plated in a density of 50,000 cells/cm² on poly-L-lysine coated coverslips in 24-well plates (Sarstedt, USA) and were held in culture for up to 14 days.

Calcium imaging. Measurements of [Ca²⁺], in single cortical neurons were carried out using the fluorescent indicator fura-2 in combination with a monochromator-based imaging system (T.I.L.L. Photonics, Gräfelfing, Germany) attached to an inverted microscope (BX51WI, Olympus, Germany). Emitted fluorescence was collected by a CCD camera. Cells were loaded with 5 μM fura-2-AM (Molecular Probes, Leiden, The Netherlands) supplemented with 0.01% Pluronic F127 for 35 min at 20-22° C. in a standard bath solution containing (in mM): 140 NaCl, 5 KCl, 1 MgCl₂, 2 CaCl₂, 10 glucose and 10 HEPES, adjusted to pH 7.4 with NaOH. For measurements of [Ca²⁺], cells were held in standard bath solution and fluorescence was excited at 340 and 380 nm. Fluorescence intensities from single cells were acquired in intervals of 2 s or 20 s. After correction for the individual background fluorescence, the fluorescence ratio R=F₃₄₀/F₃₈₀ was calculated. Quantities for [Ca²⁺], were then calculated by the equation [Ca²⁺]ᵢ=K_{D}β(R−Rₘₐₓ)/(Rₘₐₓ−R), with K_{D}=224 nM (Grynkiewicz (1985)), β=2.64, Rₘᵢₙ=0.272 and Rₘₐₓ=1.987 obtained from single dye-loaded cells in the presence of 5 μM ionomycin added to standard bath solution or to a solution containing 10 mM EGTA instead of 2 mM CaCl₂.

For oxygen-glucose deprivation (OGD) experiments, cells were immediately transferred to a N₂-aerated chamber continuously superfused with a N₂-bubbled solution containing (in mM): 140 NaCl, 5 KCl, 1 MgCl₂, 2 CaCl₂ and 10 HEPES, adjusted to pH 7.4. All experiments were carried out at 20-22° C. All chemicals were obtained from Sigma (Germany).

Brain slice preparation. Brain slices including the hippocampus were prepared from 6 to 10 week old Stim2^{-/-} mice and wild type littermates as described earlier (Meuth (2003)). In brief, coronal sections were cut on a vibratome (Vibratome®, Series 1000 Classic, St. Louis, USA) in an ice-chilled solution containing (in mM): Sucrose, 200; PIPES, 20; KCl, 2.5; NaH₂PO₄, 1.25; MgSO₄, 10; CaCl₂, 0.5; dextrose, 10; pH 7.35 adjusted with NaOH. After preparation slices were transferred to a holding chamber continuously superfused with a solution containing NaCl 120 mM, KCl 2.5 mM, NaH₂PO₄ 1.25 mM, HEPES 30 mM, MgSO₄ 2 mM, CaCl₂ 2 mM and dextrose 10 mM. pH values were adjusted using HCl. To induce in vitro ischemic conditions the pH values were reduced from 7.25 to 6.5, and glucose concentration was lowered to 5 mM under hypoxic conditions. Restriction of O₂ was achieved by perfusion with an external solution that had been bubbled with nitrogen for at least 60 minutes prior to the recordings (Plant (2002)).

Morris Water Maze. The water maze consisted of a dark-gray circular basin (120 cm diameter) filled with water (24-26° C., 31 cm deep) made opaque by the addition of non-toxic white tempera paint. A circular platform (8 cm diameter) was placed 1 cm below the water surface in the centre of the goal quadrant, 30 cm from the wall of the pool. Distant visual cues for navigation were provided by the environment of the laboratory; proximal visual cues consisted of four different posters placed on the inside walls of the pool. Animals were transferred from their cages to the pool in an opaque cup and were released from eight symmetrically placed positions on the pool perimeter in a predetermined but not sequential order. Mice were allowed to swim until they found the platform or until 180 seconds had elapsed. In this last case, animals were guided to the platform and allowed to rest for 20 seconds. The animals were submitted to six trials per day for five days using a hidden platform at a fixed position (south-east) during the first three days (18 trials, acquisition phase) and in the opposite quadrant (north-west) for the last two days (12 trials, reversal phase). Trials 19 and 20 were defined as probe trials to analyze the precision of the spatial learning.

Elevated plus-maze test. The animals were placed in the centre of a maze with 4 arms arranged in the shape of a plus (Pellow (1986)). Specifically, the maze consisted of a central quadrangle (5×5 cm), two opposing open arms (30 cm long, 5 cm wide) and two opposing closed arms of the same size but equipped with 15 cm high walls at their sides and the far end. The device was made of opaque grey plastic and elevated 70 cm above the floor. The light intensity at the centre quadrangle was 70 lux, on the open arms 80 lux and in the closed arms 40 lux.

At the beginning of each trial, the animals were placed on the central quadrangle facing an open arm. The movements of the animals during a 5 min test period were tracked by a video camera positioned above the centre of the maze and recorded with the software VideoMot2 (TSE Systems, Bad Homburg, Germany). Post-test this software was used to evaluate the animal tracks and to determine the number of their entries into the open arms, the time spent on the open arms and the total distance travelled in the open and closed arms during the test session. Entry into an arm was defined as the instance when the mouse placed its four paws on that arm.

tMCAO model. Experiments were conducted on 10-12 wk-old Stim2^{-/-} or control chimeras according to published recommendations for research in mechanism-driven basic stroke studies (Dirnagl (2006)). Transient middle cerebral artery occlusion (tMCAO) was induced under inhalation anesthesia using the intraluminal filament (6021PK10; Doccol Company) technique. After 60 min, the filament was withdrawn to allow reperfusion. For measurements of ischemic brain volume, animals were sacrificed 24 h after induction of tMCAO and brain sections were stained with 2% 2,3,5-triphenyltetrazolium chloride (TTC; Sigma-Aldrich, Germany). Brain infarct volumes were calculated and corrected for edema as described (Kleinschnitz (2007)). Neurological function and motor function were assessed by two independent and blinded investigators 24 h after tMACO as described (Kleinschnitz (2007)).

Stroke assessment by MRI. Magnetic resonance imaging (MRI) was performed repeatedly at 24 h and 7 d after stroke on a 1.5 Tesla MR-unit (Vision Siemens, Erlangen, Germany) as described (Kleinschnitz (2007)). For all measurements, a custom made dual channel surface coil designed for examination of the mice head was used (A063HACG; Rapid Biomedical, Würzburg, Germany). The image protocol comprised a coronal T2-w sequence (slice thickness 2 mm), and a coronal 3D T2-w gradient echo CISS (Constructed Interference in Steady State; slice thickness 1 mm) sequence. MR images were visually assessed blinded to the experimental group with respect to infarct morphology and the occurrence of intracranial hemorrhage (IHC).

Statistical analysis: Statistical methods used are given in the figure legends. The figures show:

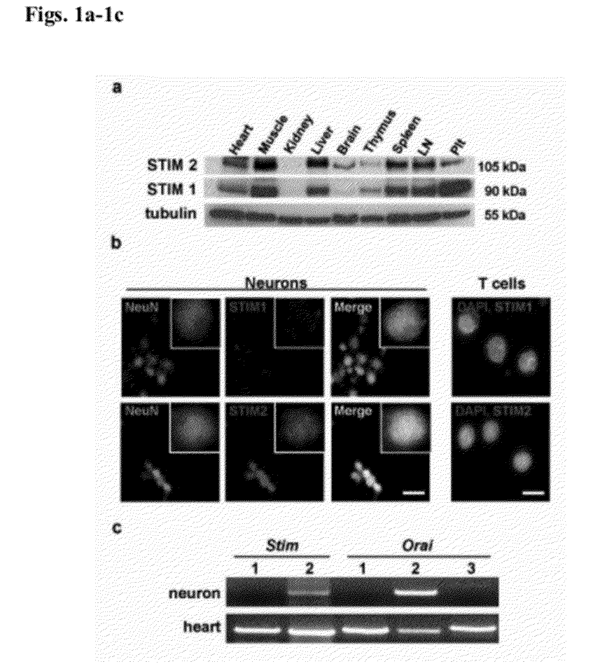

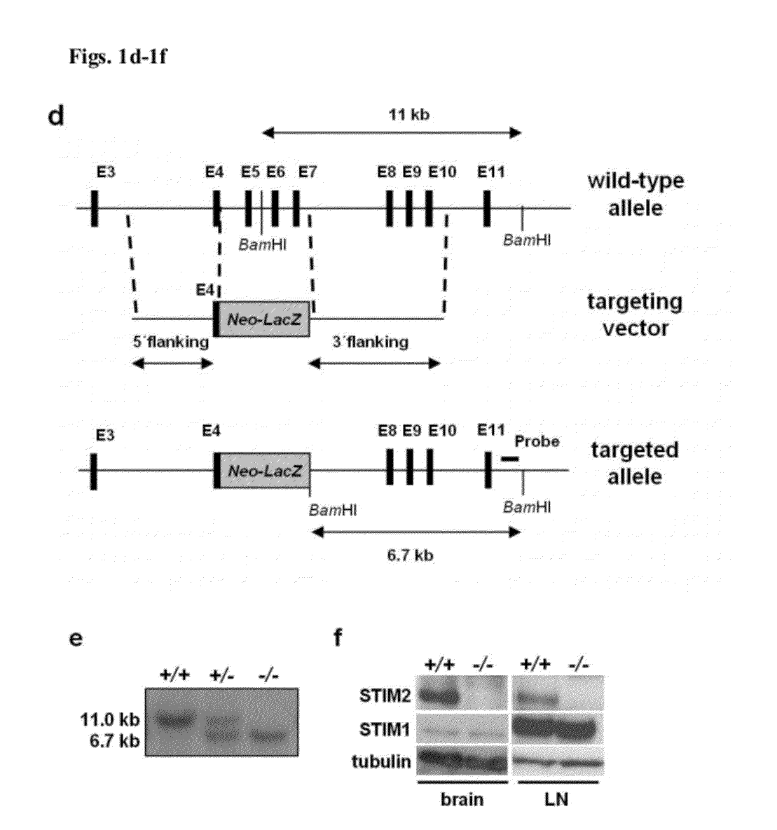

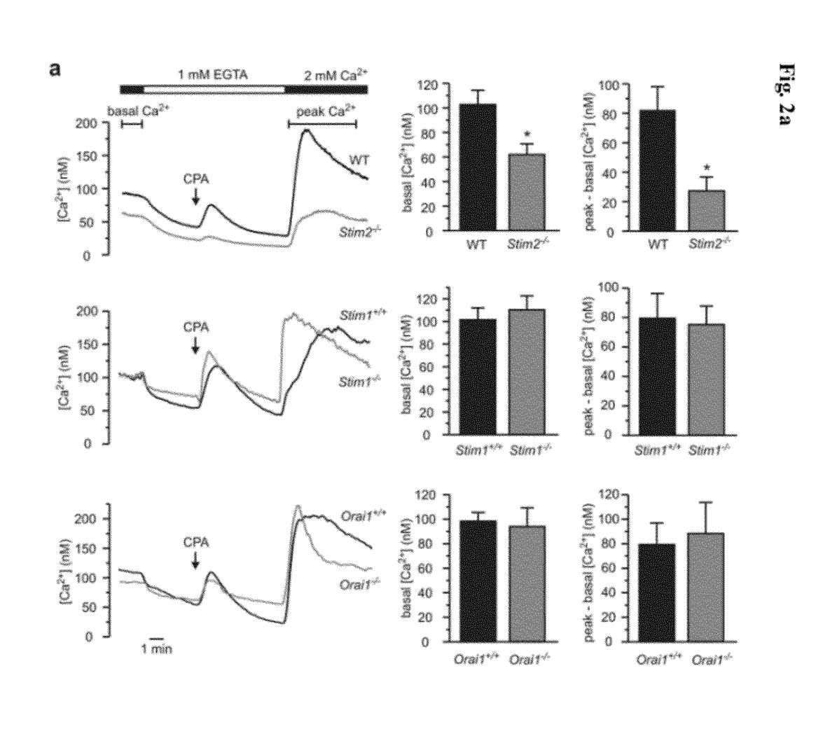

FIGS. 1a-1f: STIM2 is the main STIM isoform in neurons. a, Western blot analysis of STIM1 and STIM2 expression in different organs of 2 month old mice; α-tubulin expression was used as loading control. LN=lymph nodes, Plt=platelets b, Immunofluorescence staining of STIM1 (upper) and STIM2 (lower) of cultured hippocampal neurons (NeuN) and CD4+ T cells from wild-type mice. Cell nuclei are counterstained with DAPI. Scale bars, 100 μm (neurons), 10 μm (T-cells) c, RT-PCR of neuronal and heart cDNA using Stim and Orai primers. d, Targeting strategy for the generation of Stim2^{-/-} mice; Neo-LacZ: neomycin resistance and LacZ cassettes. e, Southern blot analysis of BamHI-digested genomic DNA of wild-type (+/+), heterozygous (+/−) or Stim2 knockout (−/−) mice labeled with the external probe. f, Western blot analysis of STIM2 and STIM1 expression in brain and lymph node (LN) cells of adult wild-type and Stim2^{-/-} mice.

FIGS. 2a-2d: STIM2 regulates Ca²⁺ homeostasis in cortical neurons. a, Neuronal cultures (DIV 5-9) were loaded with Fura-2 and averaged [Ca²⁺]ᵢ responses in Stim2^{-/-} were compared to wild-type (WT) cells, in Stim1^{-/-} compared to Stim1^{+/+} cells, and in Orai1^{-/-} compared to Orai1^{+/+} cells (n=20-35 cells each). Cells were stimulated with cyclopiazonic acid (CPA; 20 μM) followed by exchange of 1 mM EGTA for 2 mM Ca²⁺. Basal and peak [Ca²⁺]ᵢ were obtained during the time intervals indicated. b, Ca²⁺ release from intracellular stores was monitored by addition of 5 μM ionomycin in the presence of EGTA. This Ca²⁺ release was normalized to the maximum response after exchange of 1 mM EGTA for 2 mM Ca²⁺. c, Effect of combined oxygen-glucose deprivation (OGD) on [Ca²⁺]ᵢ. Cultured neurons (DIV12-14) were exposed for 1 h (WT) or 2 h (Stim2^{-/-}) to a glucose-free bath solution continuously bubbled with N₂. d, Chemical anoxia was induced in Stim2^{-/-} or WT cells by CCCP (2 μM, 30 min). Recovery from increases in [Ca²⁺]ᵢ was obtained after wash-out of CCCP for 60 min. All bars represent means of 5-7 experiments. Error bars indicate SEM. Asterisks indicate significant differences (p<0.05, two-tailed Mann-Whitney U test).

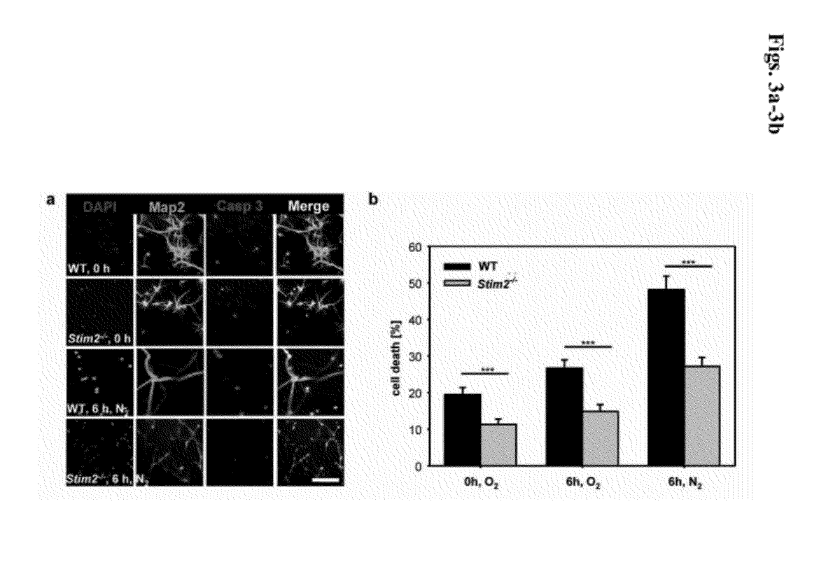

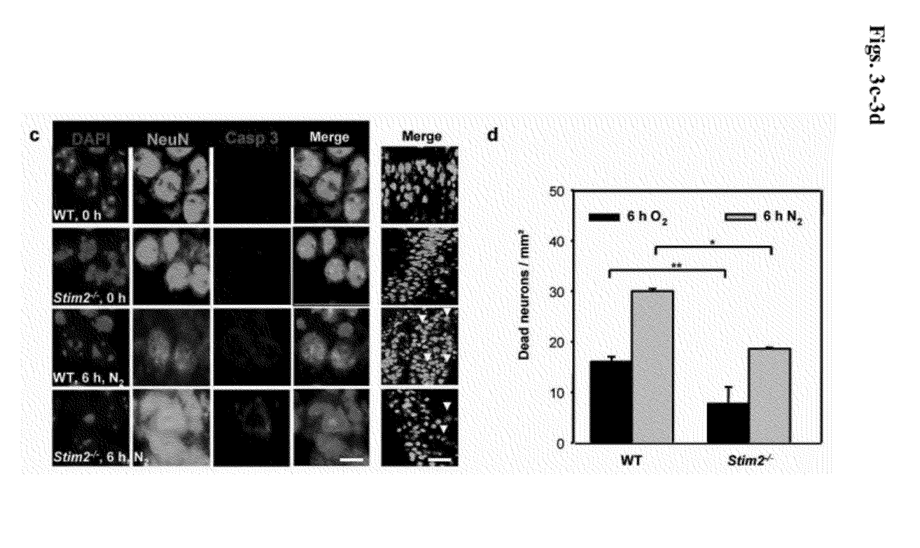

FIGS. 3a-3d: Lack of STIM2 is neuroprotective under ischemic conditions in vitro and ex vivo. a, Representative images of apoptotic (Casp3) cultured hippocampal neurons (MAP2a/b) from wild type and Stim2^{-/-} animals under control (0 h, O₂) conditions and after in vitro ischemia (6 h, N₂). Scale bar represents 50 μm. b, Bar graph representation of dead neurons (%) under the different experimental conditions. c, Representative images of caspase3 (Casp3) positive hippocampal neurons (NeuN) under ischemic and control conditions in brain slices. DAPI counterstaining (DAPI). Scale bars represent 10 (left panels) or 100 μm (right panel). d, Bar graph representation of neuronal cell death (dead neurons/mm²) under normal (6 h, O₂ black columns) and ischemic conditions (6 h, N₂, grey columns) in Stim2^{-/-} mice and control littermates. The results are presented as mean±s.d. *p<0.05, **p<0.01, ***p<0.001, using a modified student's t-test.

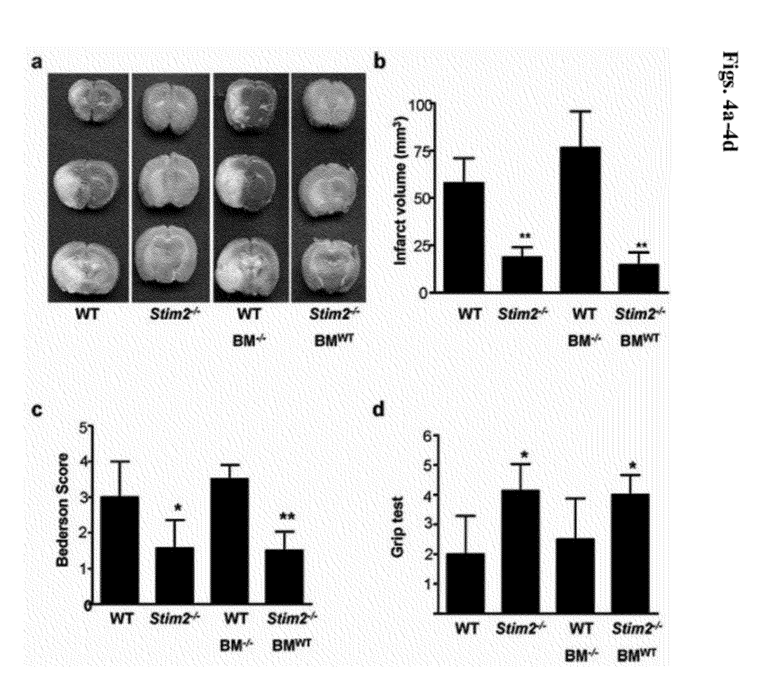

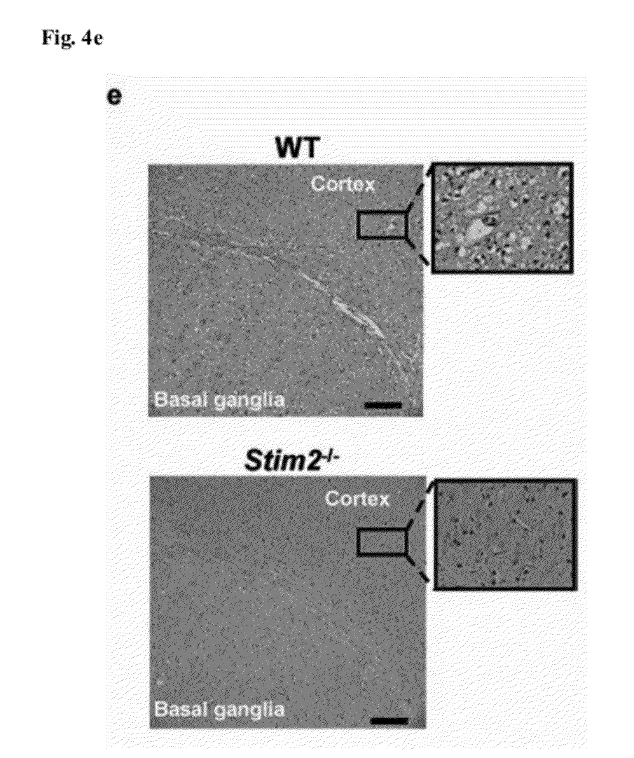

FIGS. 4a-4c: Stim2^{-/-} mice are protected from neuronal damage after cerebral ischemia. a-c, Wild-type and Stim2^{-/-} mice were subjected to transient middle cerebral artery occlusion (tMCAO) and analysed after 24 h. In parallel the experiments were performed with wild-type mice transplanted with Stim2^{-/-} bone marrow (Stim2^{+/+}BM^{-/-}) and Stim2^{-/-} mice transplanted with wild-type bone marrow (Stim2^{-/-}BM^{+/+}). a, Representative TTC (2,3,5-Triphenyltetrazolium chloride) stains of three corresponding coronal brain sections of the different groups. b, Brain infarct volumes as measured by planimetry at day 1 after tMCAO (n=8-10/group). c, Neurological Bederson score and d, grip test as assessed at day 1 after tMCAO. Graphs plot mean±s.d. (n=8-10/group). *p<0.05, **p<0.01, Bonferroni-1-Way ANOVA tested against wild-type mice. e, Hematoxylin and Eosin (HE) stained sections in the ischemic hemispheres of wild-type and Stim2^{-/-} mice. Note that infarcts are restricted to the basal ganglia in Stim2^{-/-} mice, but consistently include the neocortex in the wild-type. Scale bars, 300 μm.

FIGS. 5a-5b: Characterization of Stim2^{-/-} mice. a, Mendelian birthrate of Stim2^{-/-} mice as determined in 3-4 week old litters. b, Representative picture of an 8 weeks old male Stim2^{-/-} (−/−) mouse and a littermate control mouse (+/+).

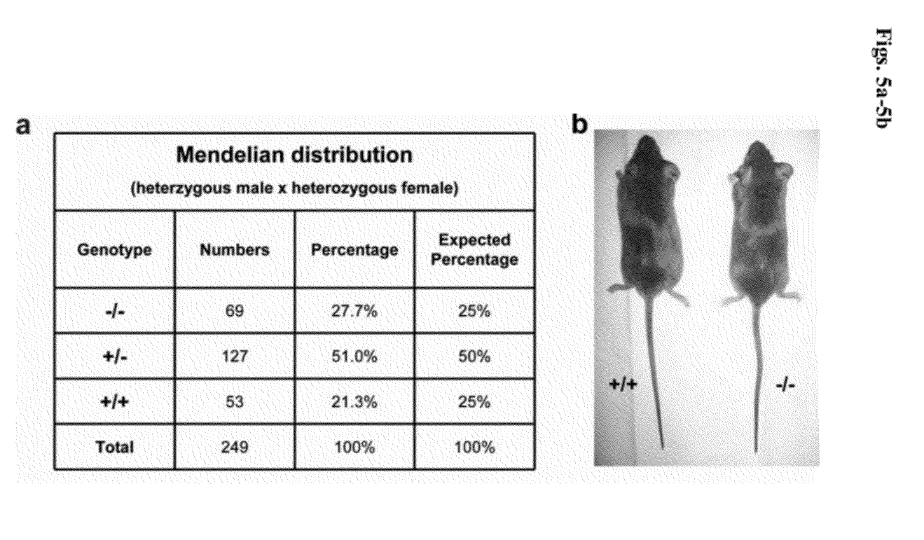

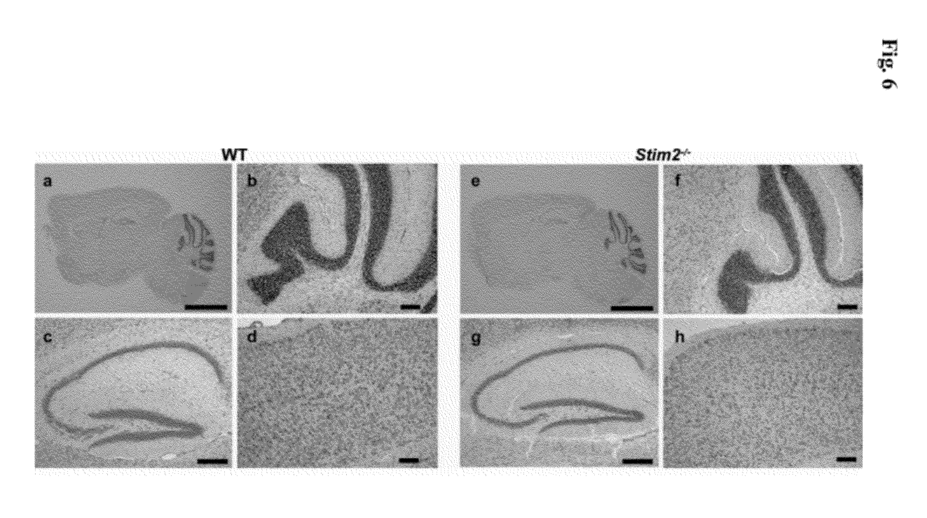

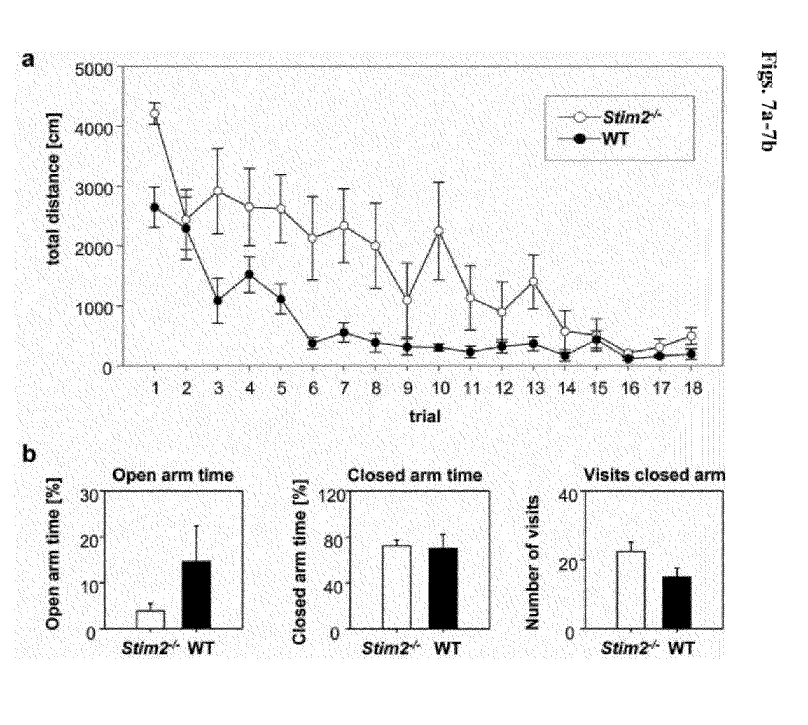

FIGS. 6a-6h: Normal brain structure in Stim2^{-/-} mice. Representative Nissl staining of 5 μm sagittal paraffin brain sections of three months old wild-type (a-d) and Stim2^{-/-} (e-h) mice (n=5 each). Macroscopic view of wild-type (a) and Stim2^{-/-} (e) brains. Higher magnification of different brain areas: cerebellum (b, 0, hippocampus (CA1, CA2, CA3 fields and dentate gyrus) (c, g), frontal pole and somatomotor areas of the neocortex (d, h). Scale bars=2.5 mm (a, e) and 250 μm (b-d, f-h).

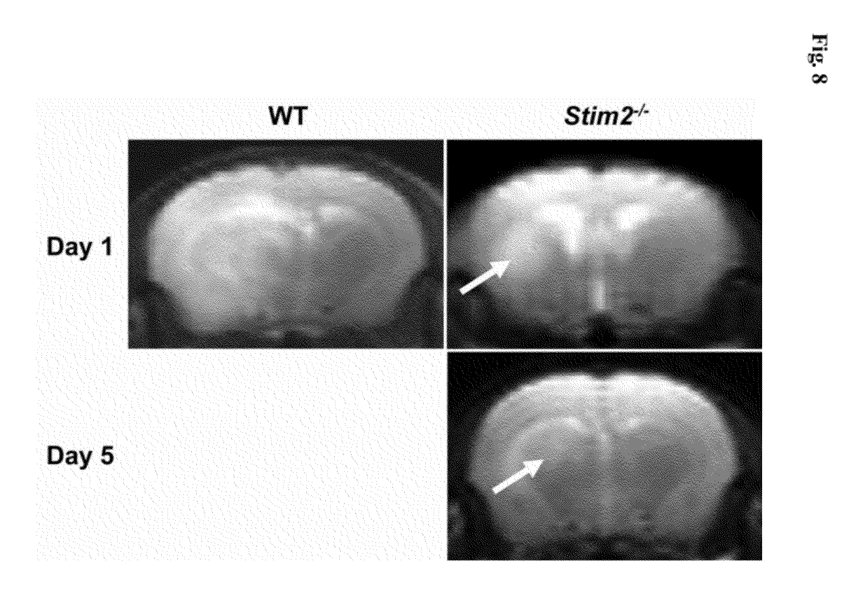

FIGS. 7a-7b: Cognitive defects of Stim2^{-/-} miceStim2^{-/-} and their wildtype littermates in the Morris Water Maze task, a standard procedure for assessing spatial and related forms of learning and memory. a, represents the total distance of swimming before the mice found the hidden platform. Values are shown as mean±SEM, F (1,12)=19.73, p<0.001, ANOVA for repeated measures. b, Stim2^{-/-} and their wildtype littermates in the elevated plus maze as an anxiety-related paradigm. Values are shown as mean±SEM, Student's t-test, n.s. The two panels on the left handed side represent the amount of time in percentage mice spent either in the open or closed arm. The last panel displays the percentages of total visits of the closed arms.

FIG. 8: Sustained neuroprotection after tMCAO in Stim2^{-/-} mice. Representative coronal T2-w MR brain images of wild-type or Stim2^{-/-} mice at day 1 and 5 after tMCAO. Infarcts are indicated by white arrows. Due to the severity of the brain damage, all wild-type mice were killed on day 1 and were, therefore, not assessed by MRI on day 5.

### Example 2

### STIM2 is the dominant STIM Isoform in Neurons

To assess the function of STIMs in mouse brain, their expression was tested by western blot analysis and a clear signal for STIM2 was obtained, whereas STIM1 was hardly detectable. In no other tested organ a comparable dominant expression of STIM2 was seen (FIG. 1a). Immunocytochemistry yielded hardly any signal for STIM1 in cultured hippocampal neurons, whereas strong perinuclear staining was detectable with anti-STIM2 antibodies, consistent with the expected ER localization of the protein (FIG. 1b). In contrast, in T cells the ratio of STIM1/STIM2 expression was reversed (FIG. 1b) (Oh-Hora (2008)). Reverse transcriptase (RT)-PCR analysis of primary neurons isolated by laser capture microscopy confirmed that STIM2 is the predominant member of the STIM family (FIG. 1c), indicating that it might have a role in Ca²⁺ homeostasis in these cells. Similar to Stim1, Orai1 mRNA was hardly detectable whereas strong and moderate expression was seen for Orai2 and Orai3, respectively (FIG. 1c).

### Example 3

### Generation of STIM2-Deficient Mice

To assess STIM2 function in vivo, the Stim2 gene was disrupted in mice (FIG. 1d, e). Mice heterozygous for the STIM2-null mutation were apparently healthy and had a normal life expectancy (not shown). Intercrossings of these animals yielded Stim2^{-/-} mice at the expected mendelian ratio, which developed normally to adulthood and were fertile (FIG. 5). Western blot analyses confirmed the absence of STIM2 in brain and lymph node (LN) whereas STIM1 expression levels were unaltered (FIG. 1f). Histological examination of major organs from Stim2^{-/-} mice showed no obvious abnormalities (not shown). Stim2^{-/-} mice did not exhibit any apparent neurological deficits and Nissl-staining on brain sections revealed no obvious structural abnormalities (FIG. 6). However, a pronounced cognitive defect became evident when the animals were subjected to the Morris Water Maze Task, the standard test for hippocampus-dependent spatial memory (Morris (1984)). Stim2^{-/-} mice had a higher latency to find the hidden platform (not shown) and the total distance moved was increased in acquisition (FIG. 7), but not in reversal trials (not shown). In contrast, examination of anxiety levels by the Elevated Plus Maze Task showed no differences between Stim2^{-/-} and wild-type littermates (p>0.05; FIG. 7). Thus, lack of STIM2 leads to a distinct cognitive impairment, possibly due to altered Ca²⁺ homeostasis in brain neurons.

### Example 4

### STIM2 regulates CCE and Ischemia-Induced Ca²⁺ Accumulation in Neurons

To test the effect of STIM2-deficiency on neuronal Ca²⁺ homeostasis directly, Ca²⁺ imaging experiments were performed in neuronal cultures extracted from cortical tissue. During these studies, it was noted that Stim2^{-/-} cultures consistently contained a higher percentage of vital cells at early culture stages (DIV 1-2) and displayed improved survival at late culture stages (>DIV 10) compared to wild-type (not shown). To assess CCE, Ca²⁺ store release was induced by the SERCA pump inhibitor cyclopiazonic acid (CPA) in the absence of extracellular Ca⁺. This treatment caused a transient Ca²⁺ signal, followed by a second [Ca²⁺]ᵢ increase after addition of external Ca²⁺ which exceeded the basal Ca²⁺ level, suggesting the presence of CCE (FIG. 2a). Strikingly, CCE was severely reduced in Stim2^{-/-} neurons compared to wild-type controls (27±9 nM vs. 82±16 nM; n=7; p<0.05; FIG. 2a) whereas no significant alterations were found in neurons from STIM1-deficient (Burnashev, 2005) and Orai1-deficient (Braun (2008)) mice compared to the respective controls (FIG. 2a). Thus, CCE in cultured neurons is regulated by STIM2 but does not require STIM1 or Orai1, the essential components of CCE in non-excitable cells.

Brandman et al. have shown that STIM2 regulates basal Ca²⁺ concentrations in the cytosol and the ER of different non-neuronal cell lines (Brandman (2007)). In line with this report, Stim2^{-/-} neurons displayed lower basal cytosolic Ca²⁺ levels than wild-type cells (62±9 nM vs. 103±12 nM; n=7; p<0.05), whereas no alteration was seen in Stim1^{-/-} and Orai1^{-/-} cells (FIG. 2a). In order to evaluate the Ca²⁺ content of intracellular stores, the release from this compartment was measured by application of the membrane permeant calcium ionophore ionomycin in the absence of extracellular Ca²⁺ (Brandman (2007)). The amplitude of the relative Ca²⁺ peak was decreased in Stim2^{-/-} cells (0.12±0.02 vs. 0.33±0.07; n=5; p<0.05), whereas the subsequent Ca²⁺ entry induced by re-addition of extracellular Ca²⁺ was indistinguishable between Stim2^{-/-} and control neurons (FIG. 2b). Thus, STIM2 also regulates basal Ca²⁺ concentrations in the cytosol and intracellular stores of murine cortical neurons.

During brain ischemia, an excessive increase in [Ca²⁺]ᵢ is thought to be a main activator of neuronal cell death (Lipton (1999)). To test a possible role of CCE in this process, Ca²⁺ imaging experiments were performed on wild-type and Stim2^{-/-} neuronal cultures under conditions of oxygen-glucose deprivation (OGD), an established system for the examination of calcium-dependent and calcium-independent mechanisms in neuronal injury (Goldberg (1993), Aarts (2003)). OGD was reported to trigger cumulative increases in [Ca²⁺]ᵢ that were mostly reversible when the duration of the insult was limited to 1 hour (Aarts (2003)). We could confirm a robust [Ca²⁺]ᵢ rise in the wild-type cultures (131±46 nM; n=5) but found only a very small increase during a 1 hour OGD in Stim2^{-/-} cells (11±15 nM; n=5; p<0.05; FIG. 2c). In Stim2^{-/-} cultures, a marked [Ca²⁺]ᵢ increase was only visible when OGD was extended to 2 hours. In the presence of the mitochondrial uncoupling agent carbonyl cyanide m-chlorophenylhydrazone (CCCP), a rapid and excessive [Ca²⁺]ᵢ increase was detectable in both wild-type and Stim2^{-/-} neurons (FIG. 2d). However, after 1 h washout of CCCP, the cytosolic Ca²⁺ level in Stim2^{-/-} cells declined more efficiently than in wild-type cells (89±3% vs. 72±3%; n=5; p<0.05; FIG. 2d).

Thus, ischemia-induced [Ca²⁺]ᵢ increases develop slower and recover faster in Stim2^{-/-} neurons compared to wild-type, which may in part be explained by the lower store content in these cells. It has been reported that anoxia causes slow depletion of the ER Ca²⁺ store and that SERCA plays a major role in cytosolic Ca²⁺ clearance in sensory neurons (Henrich (2008)). Hence, a lower ER Ca²⁺ content would decrease the cytosolic Ca²⁺ load from there and might also facilitate SERCA-mediated Ca²⁺ recovery under post-ischemic conditions.

The marked impairment of OGD-induced Ca²⁺ accumulation in Stim2^{-/-} neurons indicated a possible neuroprotective effect of STIM2 deficiency under ischemic conditions. To test this directly, cultured hippocampal neurons were subjected to OGD (FIG. 3a, b). After five to seven days under control culture conditions, 80.6±4.4% (n=5) wild-type neurons were viable, which is in agreement to previous reports (Kim (2008)). A comparable value was found for Stim1^{-/-} neurons (76.7±7.8%; n=5; p>0.05, not shown) while neurons prepared from Stim2^{-/-} mice showed increased viability (88.7±2.6%; n=3; p<0.01). After 6 h under ischemic conditions (low glucose, N₂, pH 6.5) (Plant (2002)), a similarly strong decrease in viability was seen in wild-type and Stim1^{-/-} neurons (51.9±8.4 vs. 52.4±6.6%, n=5, p>0.05, not shown) whereas Stim2^{-/-} neurons survived significantly better (72.9±4.3%; n=3, p<0.001).

Neuronal death was also monitored under ischemia-like conditions in mouse hemi-brain slices (FIG. 3c, d). After 6 h, slices from wild-type mice kept under control conditions (normoglycemia, O₂, pH 7.25) showed 16.0±1.7 dead neurons per mm² whereas this number was increased in consecutive slices that were kept under ischemic conditions (hypoglycemia, N₂, pH 6.5; 30±0.9; n=3, p<0.01). In contrast, less dead neurons were found in slices from Stim2^{-/-} mice under control (7.7±5.7, n=3, p<0.05) and ischemic conditions (18.6±0.4, n=3, p<0.05).

### Example 5

### Stim2^{-/-} Mice are Protected from Ischemic Stroke

To determine the in vivo relevance of the above findings, the development of neuronal damage was studied in Stim2^{-/-} mice in a model of transient focal cerebral ischemia with one hour occlusion of the middle cerebral artery (MCA) (Choudhri (1998)). After 24 h, infarct volumes in Stim2^{-/-} mice were reduced to <40% compared to wild-type as assessed by 2,3,5-triphenyltetrazolium chloride (TTC) staining (18.6±5.5 vs. 57.9±13.1 mm³, p<0.01) (FIG. 4a, b). Reductions in infarct size were functionally relevant, as the Bederson score assessing global neurological function (1.6±0.8 vs 3.0±1.0, respectively; p<0.05) and the grip test, which specifically measures motor function and coordination (2.0±1.3 versus 4.1±0.9, respectively; p<0.05), were significantly better in Stim2^{-/-} mice compared to controls (FIG. 4c,d). Serial magnetic resonance imaging (MRI) on living mice up to day 5 showed that infarct volume did not increase over time in Stim2^{-/-} mice, indicating a sustained protective effect (supplementary FIG. 4). In line with this, histological analysis revealed infarctions restricted to the basal ganglia in Stim2^{-/-} mice (FIG. 4e). In contrast, wild-type mice reconstituted with Stim2^{-/-} bone marrow developed regular infarcts, while infarctions remained small in Stim2^{-/-} mice transplanted with wild-type bone marrow (FIG. 4a-d). These results show that STIM2-deficiency protects mice from ischemic neuronal damage independently of functional alterations within the haematopoietic system.

### Example 6

### Materials and Methods

Mice. Orai2 (or Orai3) knockout mice are generated by disrupting essential exons of the Orai2 (or Orai3) genes, respectively, using standard molecular techniques. Orai2^{-/-} (or Orai3^{-/-}) ES cells are injected in C57B1/6 blastocysts to generate chimeric mice. Chimeric mice are crossed back with C57B1/6 (Harlan Laboratories) and subsequent progenies were intercrossed to obtain Orai2^{-/-} (or Orai3^{-/-}) mice, respectively. Genotypes are determined by PCR analysis using appropriate primer pairs.

For the generation of bone marrow chimeras, 5-6 weeks old wild-type and Orai2^{-/-} (or Orai3^{-/-}) female mice are irradiated with a single dose of 10 Gy, and bone marrow cells from 6 weeks old wild-type or Orai2^{-/-} (or Orai3^{-/-}) mice from the same litter are injected intravenously into the irradiated mice (4×10⁶ cells/mouse).

The NCBI accession numbers for the mRNA sequences of the mice proteins ORAI2 and ORAI3 are AM712356 Mus musculus ORAI calcium release-activated calcium modulator 2 (Orai2); AB271216 for ORAI calcium release-activated calcium modulator 3.

RT-PCR analysis. Mouse mRNA from 50 single neurons isolated by laser capture micro-dissection are extracted and reverse-transcribed. Orai primers are used as previously described (Braun (2008)).

Histology. Paraffin (Histolab Products AB) paraformaldehyde-fixed organs are cut into 5-μm thick sections and mounted. After removal of paraffin, tissues were stained with hematoxylin and eosin (Sigma-Aldrich) or with Nissl staining method following standard protocols.

Immunocytochemistry. Cultured hippocampal cell cultures are fixed with 4% PFA (Merck, Germany), washed 3 times with 10 mM PBS and incubated for 60 min at 4° C. in 10 mM PBS containing 5% goat serum (GS; PAA Laboratories, Austria) and 0.3% Triton X100 (Sigma, Germany). Primary antibodies (mouse MAP2a/b 1:200, abcam, United Kingdom; rabbit Cleaved Caspase-3 1:150, Cell Signaling, MA) are diluted in 10 mM PBS and incubated for 12 hours at 4° C. After washing steps with 10 mM PBS, incubation with secondary antibodies (Alexa 488 labeled goat anti-mouse 1:100, BD Bioscience, Cy-3 labeled goat anti-rabbit 1:300, Dianova, Hamburg) is carried out in the same manner. Staining with 0.5 μg/ml DAPI (Merck, Germany) is performed for 7 min. Finally, cultures were washed and subsequently covered with DABCO (Merck, Darmstadt). Pictures were collected by immunofluorescence microscopy (Axiophot; Zeiss, Jena).

Neuronal cell cultures. Neuronal cultures are obtained from Orai2^{-/-} (or Orai3^{-/-}) or control mice (E18) according to previously described preparation protocols (Meuth (2008)). In brief, pregnant mice are killed by cervical dislocation and embryos are removed and transferred into warm Hank's buffered salt solution (HBSS). After preparation of hippocampi, the tissue is collected in a tube containing 5 ml of 0.25% trypsin in HBSS. After five minutes of incubation at 37° C., the tissue is washed twice with HBSS. Thereafter, the tissue is dissociated in 1 ml of neuronal medium by triturating with fire polished pasteur pipettes of decreasing tip diameter. Neurons are diluted in neuronal medium (10% 10× modified earl's medium (MEM); 0.2205% sodium bicarbonate; 1 mM sodium pyruvate; 2 mM L-glutamine; 2% B27 supplement (all Gibco, Germany); 3.8 mM glucose; 1% penicillin/streptomycin (Biochrom AG, Germany), and plated in a density of 60,000 cells/cm² on poly-D-lysine coated cover slips in 4-well plates (Nunc, Denmark). Prior to experiments all cell cultures are incubated at 37.0° C. and 5% CO₂ and held in culture for up to 5-7 days. To induce ischemic conditions the pH is changed to 6.5, glucose concentrations are lowered and O₂ is restricted as described for the slice preparations.

For calcium measurements, primary neuronal cultures are obtained from Orai2^{-/-} (or Orai3^{-/-}) or control mice (E18-P0) as described above except for the following: Tissue was collected from whole cortices and cells are cultured in Neurobasal-A medium containing 2% B27 supplement, 1% GlutaMAX-1 and 1% penicillin/streptomycin (all Gibco, Germany). Cells were plated in a density of 50,000 cells/cm² on poly-L-lysine coated coverslips in 24-well plates (Sarstedt, USA) and are held in culture for up to 14 days.

Calcium imaging. Measurements of [Ca²⁺]ᵢ in single cortical neurons are carried out using the fluorescent indicator fura-2 in combination with a monochromator-based imaging system (T.I.L.L. Photonics, Gräfelfing, Germany) attached to an inverted microscope (BX51WI, Olympus, Germany). Emitted fluorescence is collected by a CCD camera. Cells are loaded with 5 μM fura-2-AM (Molecular Probes, Leiden, The Netherlands) supplemented with 0.01% Pluronic F127 for 35 min at 20-22° C. in a standard bath solution containing (in mM): 140 NaCl, 5 KCl, 1 MgCl₂, 2 CaCl₂, 10 glucose and 10 HEPES, adjusted to pH 7.4 with NaOH. For measurements of [Ca²⁺]ᵢ cells are held in standard bath solution and fluorescence was excited at 340 and 380 nm. Fluorescence intensities from single cells are acquired in intervals of 2 s or 20 s. After correction for the individual background fluorescence, the fluorescence ratio R=F₃₄₀/F₃₈₀ is calculated. Quantities for [Ca²⁺]ᵢ are then calculated by the equation [Ca²⁺]ᵢ=K_{D}β (R−Rₘᵢₙ)/(Rₘₐₓ−R), with K_{D}=224 nM (Grynkiewicz (1985)), β=2.64, Rₘᵢₙ=0.272 and Rₘₐₓ=1.987 obtained from single dye-loaded cells in the presence of 5 μM ionomycin added to standard bath solution or to a solution containing 10 mM EGTA instead of 2 mM CaCl₂.

For oxygen-glucose deprivation (OGD) experiments, cells are immediately transferred to a N₂-aerated chamber continuously superfused with a N₂-bubbled solution containing (in mM): 140 NaCl, 5 KCl, 1 MgCl₂, 2 CaCl₂ and 10 HEPES, adjusted to pH 7.4. All experiments are carried out at 20-22° C. All chemicals are obtained from Sigma (Germany).

Brain slice preparation. Brain slices including the hippocampus are prepared from 6 to 10 week old Orai2^{-/-} (or Orai3^{-/-}) mice and wild type littermates as described earlier (Meuth (2003)). In brief, coronal sections are cut on a vibratome (Vibratome®, Series 1000 Classic, St. Louis, USA) in an ice-chilled solution containing (in mM): Sucrose, 200; PIPES, 20; KCl, 2.5; NaH₂PO₄, 1.25; MgSO₄, 10; CaCl₂, 0.5; dextrose, 10; pH 7.35 adjusted with NaOH. After preparation, slices are transferred to a holding chamber continuously superfused with a solution containing NaCl 120 mM, KCl 2.5 mM, NaH₂PO₄ 1.25 mM, HEPES 30 mM, MgSO₄ 2 mM, CaCl₂ 2 mM and dextrose 10 mM. pH values are adjusted using HCl. To induce in vitro ischemic conditions the pH values are reduced from 7.25 to 6.5, and glucose concentration is lowered to 5 mM under hypoxic conditions. Restriction of O₂ is achieved by perfusion with an external solution that is bubbled with nitrogen for at least 60 minutes prior to the recordings (Plant (2002)).

tMCAO model and stroke assessment by MRI. These experiments are carried out as described above for the analysis of Stim2^{-/-} mice.

### Example 7

### Generation of ORAI2 and ORAI3-Deficient Mice

To assess STIM2 function in vivo, the Orai2 (or Orai3) genes is disrupted in mice. Mice heterozygous for the Orai2-(or Orai3)-null mutation are expected to be apparently healthy and to have a normal life expectancy. Intercrossings of these animals are performed to yield Orai2^{-/-} (or Orai3^{-/-}) mice Western blot analyses are performed to confirm the absence of ORAI2 (or ORAI3) in different organs. Histological examination of major organs Nissl-staining on brain sections from Orai2^{-/-} (or Orai3^{-/-}) will be carried out.

### Example 8

### ORAI2 and ORAI3 Regulates CCE and Ischemia-Induced Ca²⁺ Accumulation in Neurons

To test the effect of ORAI2 (or ORAI3)-deficiency on neuronal Ca²⁺ homeostasis directly, Ca²⁺ imaging experiments are performed in neuronal cultures extracted from cortical tissue. To assess CCE, Ca²⁺ store release is induced by the SERCA pump inhibitor cyclopiazonic acid (CPA) in the absence of extracellular Ca²⁺. It is expected that CCE is significantly reduced in Orai2^{-/-} (and Orai3^{-/-}) neurons compared to wild-type controls.

To test the role of ORAI2-(or ORAI3)-mediated CCE in this in ischemia-induced calcium accumulation in neurons, Ca²⁺ imaging experiments are performed on wild-type and Orai2^{-/-} (or Orai3^{-/-}) neuronal cultures under conditions of oxygen-glucose deprivation (OGD), an established system for the examination of calcium-dependent and calcium-independent mechanisms in neuronal injury (Goldberg (1993), Aarts (2003)). OGD was reported to trigger cumulative increases in [Ca²⁺]ᵢ that were mostly reversible when the duration of the insult was limited to 1 hour (Aarts (2003)). It is expected that only a very small increase during a 1 hour OGD in Orai2^{-/-} (and Orai3^{-/-}) cells.

To test a possible neuroprotective effect of ORAI2 (or ORAI3) deficiency under ischemic conditions, cultured hippocampal neurons are subjected to OGD. After five to seven days under control culture conditions the cells are exposed for 6 h to ischemic conditions (low glucose, N₂, pH 6.5) (Plant (2002)). It is expected that Orai2^{-/-} (and Orai3^{-/-}) neurons survive this ischemic insult significantly better than wild type control neurons.

Neuronal death is also monitored under ischemia-like conditions in mouse hemi-brain slices. After 6 h under ischemic conditions (hypoglycemia, N₂, pH 6.5; 30±0.9), slices from Orai2^{-/-} (and Orai3^{-/-}) mice are expected to show significantly less dead neurons than slices from wild-type mice.

### Example 9

### Orai2^{-/-} (and Orai3^{-/-}) Mice are Expected to be Protected from Ischemic Stroke

To determine the in vivo relevance of the above findings, the development of neuronal damage is studied in Orai2^{-/-} (or Orai3^{-/-}) mice in a model of transient focal cerebral ischemia with one hour occlusion of the middle cerebral artery (MCA) (Choudhri (1998)). After 24 h, infarct volumes in Orai2^{-/-} (and Orai3^{-/-}) mice are expected to be significantly reduced compared to wild-type as assessed by 2,3,5-triphenyltetrazolium chloride (TTC) staining Reductions in infarct size are expected to be functionally relevant, as tested by the Bederson score assessing global neurological function and the grip test, which specifically measures motor function and coordination. Serial magnetic resonance imaging (MRI) on living mice up to day 5 are expected to show that infarct volume does not increase over time in Orai2^{-/-} (and Orai3^{-/-}) mice, indicating a sustained protective effect. Histological analysis is expected to reveal infarctions restricted to the basal ganglia in Orai2^{-/-} (and Orai3^{-/-}) mice.

### REFERENCES

Aarts, M. et al; Cell 115, 863-877 (2003)
Berridge, M. J., Bootman, M. D., Roderick, H. L.; Nat. Rev. Mol. Cell. Biol. 4, 517-529 (2003)
Berridge, M. J.; Neuron 21, 13-26 (1998)
Brandman, O., Liou, J., Park, W. S., Meyer, T.; Cell 131, 1327-1339 (2007)
Braun, A. et al.; Blood, in press (2008) [epub ahead of print October 02]
Burnashev N. and Rozov, A.; Cell Calcium 37, 489-495 (2005)
Choudhri, T. F. et al; J Clin Invest 102, 1301-1310 (1998)
Dirnagl, U.; J. Cereb. Blood Flow Metab 26, 1465-1478 (2006).
Emptage, N. J., Reid, C. A., Fine, A.; Neuron 29, 197-208 (2001)
Feske, S. et al.; Nature 441, 179-185 (2006)
Frandsen, A. and Schousboe, A.; J. Neurochem. 56, 1075-1078 (1991)
Goldberg, M. P. and Choi, D. W.; J. Neurosci. 13, 3510-3524 (1993)
Grynkiewicz, G., Poenie, M., Tsien, R. Y.; J. Biol. Chem. 260, 3440-3450 (1985).
Henrich, M. and Buckler, K. J.; J. Neurophysiol. 100, 456-473 (2008)
Helmchen, F., Imoto, K., Sakmann, B.; Biophys. J. 70, 1069-1081 (1996)
Kim, H. J, Martemyanov, K. A., Thayer, S. A.; J. Neurosci. 28, 12604-12613 (2008)
Kleinschnitz, C. et al.; Circulation 115, 2323-2330 (2007).
Liou, J. et al., Curr. Biol. 15, 1235-1241 (2005)
Lipton, P.; Physiol Rev. 79, 1431-1568 (1999)
Mattson, M. P.; Aging Cell 6, 337-350 (2007)
Morris, R.; J. Neurosci. Methods 11, 47-60 (1984)
Mattson, M. P., Zhu, H., Yu, J., Kindy, M. S.; J. Neurosci. 20, 1358-1364 (2000)
Meuth, S. G. et al; J. Neuroimmunol. 194, 62-69 (2008).
Meuth, S. G. et al.; J. Neurosci. 23, 6460-6469 (2003).
Morris, R. G., Anderson, E., Lynch, G. S., Baudry, M.; Nature 319, 774-776 (1986)
Oh-Hora, M. et al., Nat. Immunol. 9, 432-443 (2008)
Pellow, S. and File, S. E.; Pharmacol. Biochem. Behay. 24, 525-529 (1986).
Plant, L. D., Kemp, P. J., Peers, C, Henderson, Z. Pearson, H. A.; Stroke 33, 2324-2328 (2002)
Putney, Jr., J. W.; Cell Calcium 34, 339-344 (2003)
Rao, V. R. and Finkbeiner, S.; Trends Neurosci. 30, 284-291 (2007)
Stiber, J. et al.; Nat. Cell Biol. 10, 688-697 (2008)
Takechi, H., Eilers, J., Konnerth, A.; Nature 396, 757-760 (1998)
Varga-Szabo, D. et al.; J. Exp. Med. 205, 1583-1591 (2008)
Vig, M. et al.; Science 312, 1220-1223 (2006)
Wojda, U., Salinska, E., Kuznicki, J.; IUBMB Life 60, 575-590 (2008)
Zhang, S. L. et al., Nature 437, 902-905 (2005)

1. A pharmaceutical composition comprising: an inhibitor of stromal interaction molecule 2 (STIM2) or an inhibitor of STIM2-regulated plasma membrane calcium channel activity and optionally a pharmaceutically acceptable carrier, excipient and/or diluent.

2. An agent comprising, an inhibitor of stromal interaction molecule 2 (STIM2) or an inhibitor of STIM2-regulated plasma membrane calcium channel activity useful for the treatment or prevention of a neurological disorder associated with pathologically increased cytosolic calcium concentrations.

3. A method of treating or preventing a neurological disorder associated with pathologically increased cytosolic calcium concentrations comprising: administering a pharmaceutically effective amount of an inhibitor of stromal interaction molecule 2 (STIM2) or of an inhibitor of STIM2-regulated plasma membrane calcium channel activity to a subject in need thereof.

4. The pharmaceutical composition of claim 1 wherein the inhibitor is an antibody, an antibody fragment or derivative thereof, an aptamer, a siRNA, a shRNA, a miRNA, a ribozyme, an antisense nucleic acid molecule, modified versions of these inhibitors or a small molecule.

5. The pharmaceutical composition of claim 1 wherein the STIM2-regulated plasma membrane calcium channel activity is one or more of ORAI1, ORAI2, ORAI3 or a TRP channel.

6. The pharmaceutical composition of claim 1, further comprising one or more of a neuroprotective, or a neuroregenerative substance or an antithrombotic substance.

7. The pharmaceutical composition of claim 6, wherein the one or more of a neuroprotective or a neuroregenerative substance is selected from the group consisting of a glutamate antagonist, a glutamate receptor antagonist, a glutamate release inhibitor, an antioxidant, a free-radical reducing agent, a calcium antagonist, a calcium channel blocker, a calcium chelator, a potassium channel activator, a GABA agonist, an opiate antagonist, a leukocyte adhesion inhibitor, an inhibitor of cytokines, a membrane stabilizer, a neutrophil modulator, a glycine antagonist, an apoptosis modulator, a neuronal guidance modulator, a neurotrophic factor and a stem cell modulator.

8. The pharmaceutical composition of claim 6, wherein the antithrombotic substance is a recombinant tissue plasminogen activator.

9. The agent of claim 2, wherein the neurological disorder associated with pathologically increased cytosolic calcium concentrations is selected from cerebral ischemia, brain stroke, ischemic stroke, hemorrhagic stroke, Alzheimer's disease, Parkinson's disease, Huntington's disease, autosomal dominant spinocerebellar ataxias, glaucoma, amyotrophic lateral sclerosis, epilepsy, schizophrenia, traumatic brain injury and HIV dementia.

10. A pharmaceutical composition comprising, an inhibitor of stromal interaction molecule 2 (STIM2) or an inhibitor of STIM2-regulated plasma membrane calcium channel activity; and one or more of a neuroprotective, a neuroregenerative substance, an antithrombotic substance; and optionally, a pharmaceutically active carrier, excipient or diluent for simultaneous, separate or sequential use in therapy.

11. The pharmaceutical composition of claim 10 for treating or preventing a neurological disorder associated with pathologically increased cytosolic calcium concentrations or as a lead compound for developing a drug for one or more of treating or preventing a neurological disorder associated with pathologically increased cytosolic calcium concentrations.

12. A method of identifying a compound suitable as a lead compound or as a medicament for treatment or prevention of a neurological disorder associated with pathologically increased cytosolic calcium concentrations, comprising the steps of:
a) determining the level of STIM2 protein or Stim2 transcript in a cell;
b) contacting said cell or a cell of the same cell population with a test compound;
c) determining the level of STIM2 protein or Stim2 transcript in said cell after contacting with the test compound; and
d) comparing the level of STIM2 protein or Stim2 transcript determined in step (c) with the STIM2 protein or Stim2 transcript level determined in step (a), wherein a decrease of STIM2 protein or Stim2 transcript level in step (c) as compared to step (a) indicates that the test compound is a compound suitable as a lead compound or as a medicament for the treatment or prevention of a neurological disorder associated with pathologically increased cytosolic calcium concentrations.

13. A method of identifying a compound suitable as a lead compound or as a medicament for one or more of a treatment for or prevention of a neurological disorder associated with pathologically increased cytosolic calcium concentrations, comprising the steps of:
a) emptying intracellular calcium stores of a cell containing STIM2 protein in absence of extracellular calcium and determining increase in intracellular calcium concentration upon addition of extracellular calcium;
b) contacting said cell or a cell of the same cell population containing STIM2 protein with a test compound;
c) after contacting with the test compound, emptying the intracellular calcium stores of the cell of (b) in the absence of extracellular calcium and determining the increase in intracellular calcium concentration upon addition of extracellular calcium in said cell; and
d) comparing the increase in intracellular calcium concentration determined in step (c) with the increase in intracellular calcium concentration determined in step (a), wherein no increase in intracellular calcium concentration or a smaller increase in intracellular calcium concentration in step (c) as compared to step (a) indicates that the test compound is a compound suitable as a lead compound or as a medicament for one or more of the treatment for or prevention of a neurological disorder associated with pathologically increased cytosolic calcium concentrations.

14. The method of claim 12, wherein said cell comprising the STIM2 protein is a neuronal cell.


SEQUENCE LISTING (12 sequences)

SEQ ID NO: 1 - DNA, 3924 bp, Homo sapiens
ggcggagcgtggtactacgaccagcgcgggccggagggggcggggggatgcgccgcggcggcggcggcgcgggagctggggttggtgtttggcggcgccagagcagcggatcccggtctcgccgcagcagcagcgcgggtgtcgtgcaccgcctgaagacgccgtacctttctaccccccaccttttttttttttttttttaaataaccggaaccaatgaacgcagccgggatcagagctccggaggccgccggtgccgatgggaccaggctggcgcccggcgggagcccgtgtctgaggcggcgggggcggccggaggagtcgccggcggcggtggtggcgcctcgcggagccggcgagctgcaggcggccggggcgccgctgcgctttcacccggcttctcctcggcgccttcatcccgcctcgactcctggcccagcgtggggctggctgctgcggcggcggcgctgggctgcgttgctggtgctcgggctgctggtagccggagcggcggacggatgcgagcttgtgccccggcacctccgcgggcggcgggcgactggctctgccgcaactgccgcctcctctcccgccgcggcggccggcgatagcccggcgctcatgacagatccctgcatgtcactgagtccaccatgctttacagaagaagacagatttagtctggaagctcttcaaacaatacataaacaaatggatgatgacaaagatggtggaattgaagtagaggaaagtgatgaattcatcagagaagatatgaaatataaagatgctactaataaacacagccatctgcacagagaagataaacatataacgattgaggatttatggaaacgatggaaaacatcagaagttcataattggacccttgaagacactcttcagtggttgatagagtttgttgaactaccccaatatgagaagaattttagagacaacaatgtcaaaggaacgacacttcccaggatagcagtgcacgaaccttcatttatgatctcccagttgaaaatcagtgaccggagtcacagacaaaaacttcagctcaaggcattggatgtggttttgtttggacctctaacacgcccacctcataactggatgaaagattttatcctcacagtttctatagtaattggtgttggaggctgctggtttgcttatacgcagaataagacatcaaaagaacatgttgcaaaaatgatgaaagatttagagagcttacaaactgcagagcaaagtctaatggacttacaagagaggcttgaaaaggcacaggaagaaaacagaaatgttgctgtagaaaagcaaaatttagagcgcaaaatgatggatgaaatcaattatgcaaaggaggaggcttgtcggctgagagagctaagggagggagctgaatgtgaattgagtagacgtcagtatgcagaacaggaattggaacaggttcgcatggctctgaaaaaggccgaaaaagaatttgaactgagaagcagttggtctgttccagatgcacttcagaaatggcttcagttaacacatgaagtagaagtgcaatactacaatattaaaagacaaaacgctgaaatgcagctagctattgctaaagatgaggcagaaaaaattaaaaagaagagaagcacagtctttgggactctgcacgttgcacacagctcctccctagatgaggtagaccacaaaattctggaagcaaagaaagctctctctgagttgacaacttgtttacgagaacgactttttcgctggcaacaaattgagaagatctgtggctttcagatagcccataactcaggactccccagcctgacctcttccctttattctgatcacagctgggtggtgatgcccagagtctccattccaccctatccaattgctggaggagttgatgacttagatgaagacacacccccaatagtgtcacaatttcccgggaccatggctaaacctcctggatcattagccagaagcagcagcctgtgccgttcacgccgcagcattgtgccgtcctcgcctcagcctcagcgagctcagcttgctccacacgccccccacccgtcacaccctcggcaccctcaccacccgcaacacacaccacactccttgccttcccctgatccagatatcctctcagtgtcaagttgccctgcgctttatcgaaatgaagaggaggaagaggccatttacttctctgctgaaaagcaatgggaagtgccagacacagcttcagaatgtgactccttaaattcttccattggaaggaaacagtctcctcctttaagcctcgagatataccaaacattatctccgcgaaagatatcaagagatgaggtgtccctagaggattcctcccgaggggattcgcctgtaactgtggatgtgtcttggggttctcccgactgtgtaggtctgacagaaactaagagtatgatcttcagtcctgcaagcaaagtgtacaatggcattttggagaaatcctgtagcatgaaccagctttccagtggcatcccggtgcctaaacctcgccacacatcatgttcctcagctggcaacgacagtaaaccagttcaggaagccccaagtgttgccagaataagcagcatcccacatgacctttgtcataatggagagaaaagcaaaaagccatcaaaaatcaaaagcctttttaagaagaaatctaagtgaactggctgacttgatggaatcatgttcaagtggcatctgtaaactattatcccccaccctccactccccaccttttttttggtttaattttaggaatgtaactccattggggctttccaggccggatgccatagtggaacatccagaagggcaactgtctactgtctgcttatttaagtgactatatataatcaattcatcaagccagttattactgaaaaatcattgaaatgagacagtttacagtcatttctgcctatttatttctgctttgttctcagtgatgtatatgcaacattttgttgaaagccacgatggacttacaagctttaatggactcgtaagccagcatgggcttgcaaaaatttcttgtttaccagagcatcttcttatctttccacagagctatttacatcctggactatataacttaaaagaagtaaaacgtaattgcactactgttttccagactggaaaaaaaaaaaatctctgcaagtgaaactgtatagagtttataaaatgactatggataggggactgttttcacttttagatcaaaatgggtttttaagtagaacctagggtttctaattgacttgatttctggaaatgaaaacccgcgcttttattatgggaagcttcttgaactgcatttactattgtgaagtttcaagtcccgctgtaaagatcatgttgttttgttttccccagggctttcactgtgatttactgcattgcaggctgtatgataaaacacacataatttaaagagagaaggctcttgattccttatgcaagtggaagagttgaaacttgattgaaggacttaaaacattcacaaccttaagccgaggtggggggatatggggattcaggcaattgtttacacactttgaataactgcaaaggatttacggtttgtgaaaaatgtgtactgtggaaaagataataaattgaagacattattgtgtgggattgtgctgatttttgttgataacacaaaaaacactatgttttctggagagctgtgtaagctgtcttgttgcttagttgcaatataagaaatagtgatgttttggacgtaagttgtcaacaaatttctattttatattgttatatttttatgtagtttgaaatgtaaaaatgttctaatatcaagattaacaaatataaatttatggtgcatttagaaaaaaaaaaaaaaaa

SEQ ID NO: 2 - DNA, 4033 bp, Mus musculus
agcggatcccggtgtcgccgcggcagctgcgcgggtgtcgcgctgggtcggaagacgccgtaccactgcgctcccccacttttttttttttttttttttttaaataaccggaaccaatgaacgcggcggcgagccgagcttcgcgggccgccggtgaagggagcgggtcagcacccggcgggagtccgcttccgaggcggccgggccccgggcagctggcggtgagcgagtggccggcggctcctgtgagcgctcctcccgccgtggcaccgggccgggcctcgggccgccctctccggcggcgctgggcagcaatgctgctcttcgggttgttggtggccggcgtggcggacggatgcgatctggtgccccggcacctccgcgggcggcgcgcgtcgggatcggccggagcggcggcgtctccttcggctgcggctgcgggcgagcgccaggcgctgctgacagatccctgtatgtcgctgagtccaccttgcttcactgaagaagacaggtttagcttggaagcacttcagacaatacacaaacagatggacgacgacaaggacggcgggatcgaagtggacgagagtgatgagtttatcagagaagatatgaaatataaagatgctacgaataaacatagtcacctgcacagagaagataagcacataactgttgaggatttgtggaaacagtggaaaacatcagaagttcacaattggacacttgaggataccctgcagtggttaatagaatttgttgaactgccacaatatgagaagaattttagggataataatgtgaagggaacaacactccccaggatagcagttcatgaaacttcatttatgatttcccagttgaaaatcagcgaccgaagtcacagacagaaactccaactcaaagccctggatgtggttctgtttgggcctctgacacgcccacctcataactggatgaaggattttattctcacaatttccatagtaattggtgttgggggttgttggtttgcttatacacaaaataagacatcaaaggaacatgttgcaaaaatgatgaaagacttagagagtctgcagactgcagagcagagtctcatggacttacaagagagacttgaaaaggcacaggaagaaaacagaactgttgctgtagaaaagcaaaatctggaacggaaaatgatggatgaaatcaactatgccaaggaggaggcctgtcggctgcgggagctgagggagggcgcagagtgtgagctgagcaggcgccagtatgcagagcaggaactggagcaggtccgcatggctctaaaaaaggccgaaaaggagtttgaactgagaagcagctggtctgtccctgacgcactacagaaatggcttcagctaacacacgaagttgaagtacagtactacaatattaagaggcaaaatgctgagatgcagctagccatcgctaaggacgaggtcgctgcctcctatctcctgcaggcagaaaaaattaaaaagaagagaagcacagtctttgggaccctgcacgttgcacacagctcctccctggacgaagtagaccacaagattctggaagccaagaaagccctctctgagctgaccacgtgcttgcgagaacggctttttcgctggcagcagattgagaagatctgtggctttcagatagctcacaactctgggctccccagtctcacctcctctctgtactctgaccacagctgggtggtgatgcctagagtctccattccaccctaccctattgctggaggagttgatgacctcgatgaagacacacccccaatcgtgccacagtttccagggaccgtggctaaacctgcaggatctttagccagaagcagtagtttatgccgctctcgtcgcagcatcgtgccatcctccccacagtctcagcgagctcagcttcctgctcatgctcctctggcagcccaccctcggcaccctcaccatccgcagcatccccagcactcgttgccttccccagatccagacatcctgtctgtgtcaagttgccctgctctgtatcggaacgaagaggaggaggaggctatctacttcactgctgagaaacaatgggaagtgccagacacagcttcagaatgtgactccttaaactcttccagtgggagaaaaccgtctcccccttcaagccttgagatgtaccaaacattgtcttcccgaaaaatctcaagagacgagctttccctggaggactcttccaggggggagtcacccgtgacagcagatgtctcccggggctcccctgagtgtgtgggtctgacggagaccaagagcatgatcttcagccctgcaagcagagtgtacaatggcatcctggagaaatcctgcagcatgcaccagctctccagcggcatcccggtgcctcatccccgacacacatcgtgctcctcagccggcaatgatagcaagccagttcaggaagcctcgaatgtttccagagtaagcagcatcccacatgacctctgtcataatggtgagaaaagcaaaaagccatccaaaatcaaaagccttttcaagaagaagtctaagtgactcggctgacgtgatggaattccattcaagtggcatctgtaaactgttatctcccaccctccactccgtttttattttacttttaggaatgtaactccactggggctttccagacttgatgccatagtggaaatgtccgtaagggcagctgtctactgtctgcttatatctaactgactacgcaatcaattcatcaagtcagataccaccaccggagccatcggcaggaggcagtttacagttctttccgcctatttatttctgcattgatgttagtgatgtacacacagcattctgtcgaaagccgccatggacttacaagctttaatggactgtaagccagcatgggcttgcaaaattttcttgtttaccagaatatcttcttatctttccacagagctgtttccatcatggactaaataacttaaaagaagtaaaactgattgcactactgttttccagactggaaaaaaaaatctctgcaagtaaaactgtatagagtttataaactgactgtggatagggaattgttttcacttttagataaaatggatttttaagtagaaactagggtttctaattgacttgatttctgaaaatgaaaacccacacttttattatgggacgcttcctcaaatgcatttattattataaagtttcaagtcctgctgtaaagaccatgttgttttgttttccccagggctttcactgtgatttactgcactgcaggctgtatgataaaatataaataatgtaaaggcagaaggctcttgattccttatccaagtggaagagtaaaacttgatcgaaggacttaaaacattcacatgcttaaactgcggtctggggcgtggggacccaggcacttgtttacagactttgaataaatgcaaagaatttatggtttgtgaaaaatttgtactgtggaaaagataataaattggagacattattgtgtggggttgtgctgatttttgttgataacactccactgaaaacactgttttctggagagccatgtaagctgtcttgctgctgaattgcagtataagaaatagcgatgttttggaactaagttgtcaacaaatttctttgttttatattgtttgtcatatttttatgtagtttgaaatctttaaatgttctcatatcaagattaacaaatataaattgctggtgcatttagattgtgttgaatgagtttgtaaagtttggaactggttagctgctgggcaggtcagagagaagcctgagagtagcctcgcataggacagcagtgcacatttcgtttccaccttctcagctcttaagacctcaggcagaactatctggaaatacaggaagatgggtggttgggtgaacacggatatgttaactagaatttttttttggtgctggctatctgggaatgctgatggataagtaagaagtccccttgagatttgcataaaagg

SEQ ID NO: 3 - DNA, 3609 bp, Mus musculus
cccgggcctggcccgtgcgcgtccgcctgctgcaccggggcaccaggagccgcagaggtaccggacccggcgggggcgctgacctcggcctaggagtcttaggatcccggagacatccgtgtctgctggggcctgtaggtcgcacgccgaagccctagctgctgaggctcaccgctgtttctcggggcgaggtcaggtgccccccttctctcctctcttctctccctcccccacctcagtgcgggcgggagattctgcccgcctcctcccgcaggggtgcagcaggctgcggagctgacagcggccccgcagccaccctgctcaaactctccggaagcagatagagctcaggccgccgccgcagccccggcggacctacagttggaccggagactccgatccttctgcgtccaaacttggggcacttgaccttcgcttatcggaggaggtaagcccgcaggtggctggacagctgcggcgccgcgagggcatcttgctttggaaccgtcggctgcactccctgactctgggatttgcttctgggatccaaaggtgtctacagcaggcgcatgttgactgagacctaccgtcatggatgtgtgcgcccgtcttgccctgtggcttctttgggggctccttctgcatcagggccagagtctcagccatagtcacagtgaaaagaatacaggagctagctccggggcgacttctgaagagtctaccgaagcagagttttgccgaattgacaagcccctgtgccacagtgaggatgagaagctcagctttgaggccgtccgaaacatccataagctgatggatgacgatgccaatggtgatgtggatgtggaagaaagtgatgagttcctaagggaagacctcaattaccatgacccaacagtgaaacatagcaccttccatggtgaggataagcttatcagcgtggaggacctgtggaaggcgtggaagtcatcagaagtgtacaactggactgtggatgaggtgatacagtggctcattacgtatgtggagctgccacagtatgaggaaaccttccggaagttgcagcttactggccacgccatgccaaggctagcagtaaccaacaccaccatgacagggactgtactgaagatgacagatcggagccacaggcagaagctgcagctgaaggccctggacacagtgctgtttgggcctcctctcttgactcggcataatcacctgaaggacttcatgctggtggtgtctatcgttattggtgtgggtggctgctggtttgcctatatccagaaccgttactctaaggagcacatgaagaaaatgatgaaggatctggaagggttacaccgggctgagcagagtctgcatgaccttcaggaaaggctgcacaaggcccaggaggagcaccgaactgtggaagtagagaaggtccacctggagaagaagctgcgagatgagatcaaccttgccaagcaggaagctcagcggctgaaggagctgagggagggtactgagaatgagaggagccgtcaaaaatatgctgaggaagagctggagcaggttcgggaggccttgaggaaagcagagaaggagctggaatcacacagctcatggtatgctcctgaggccctgcagaagtggctgcagctgacccatgaggtggaggtgcagtactacaacatcaagaagcaaaatgcagagaggcagctgctggtggccaaggagggggctgagaaaataaaaaagaagagaaacacgctttttggtaccttccatgtggcccacagctcttccctggatgatgtggatcataaaatcctaactgctaagcaagctctgagtgaggtgacagcggcactgagggagcgcctgcaccggtggcagcagatcgagatcctctgcggtttccagattgtcaataaccccggcatccactccttggtggctgctctcaacatcgaccccagctggatgggcagcacccgccctaaccccgcccacttcatcatgactgacgatgtggatgacatggatgaggagattgtgtcgcccttgtccatgcagtcccccagcctgcagagcagtgtccggcagcgcctgacggagccacagcttggcctgggatctcagagggatttgacccattccgattcggagtcctccctccacatgagtgaccgccagcgtgtggcccccaagcctcctcagatgggccgtgctgcagatgaagctctcaatgccatgccttccaatggcagccatcggctgattgagggggtccatccaggatctctggtggagaaactgcctgacagccctgctctggccaagaagacatttatggcgttgaaccatggcctagacaaggcccacagcctgatggagctgaacccctcagtcccacctggtggctccccacttttggattcttcccattctcttagccccagttccccagacccagacacgccatctccagttggggacaaccgagctctgcagggtagccgaaacacacgaattccccacttggctggcaagaaggcaatggctgaggaggataatggttccattggtgaggagacagactccagtccaggcaggaagaagtttcctctcaaaatttttaagaagcctcttaagaagtaggcagactagggtggtagtgttgagacagcctgtccttccctgggtcttctgccttcacctcccttcctttctttgcaatatctggctcctagagtggggcacagaggggctggcccaagggcctgggcactgtacatatctgccctgctcatccttggtccttcatcattatttattaactgaccaccatggcctgcctgtcgggaaacccttccacccatgggctgctgctgtcacatcttctccacttcagtgcatgtcttagttgctcttccctcagttcccactccacttttggggtccagcttctgtctctgctgtcccagttttgaggtttggtttttgtttctgtctcctgctttcaggctcctctctcccattactccccaacgatcctagcagttgtggggaagataggaggagtaacttctgacacctgtacctcagatctgttcatcctactcacagccattctgcctaccccagactgggccacggtcctgatctctggggtttgttctatggaagtgtggtagactggagggaacctcatcctggagctcctttggatctccagggctccattcagggagtggaaccaactcccagggaacaagtcaccagagttttaaagagagaccaggcttgtgattgatgggagagacttctcccagttggaggatgagtagatgccaaagctgtgggctgtaaggcagttgccatttctctcttgccctgcccacacctgccttcctcttacctctgctcccctatattgcaggagtgtatctcttaagaggtgctgccctgaagctccccatcagcatcagcactactggggctcagggcaactggctcccctggctatgggagccacagtcatgacacagggctcttgtggagccctgggcaaggatgttatatttgaaccaaaagacaaacagttttaaaataaaaaaa

SEQ ID NO: 4 - DNA, 1871 bp, Mus musculus
gcggccgcttggaacttgcggagcggttctgcagccagtgcctcggccttcggatccggtgcgtccggctgcgggagaccggctcgagtgggaggccggcggaggtgcggcgcggcccggcccggccccggggccgcagccttctcaggcgctcgccgccagccgcgccttcgcagcgtactccatgcatccggagcctgccccgcccccgagtcacagcaatccggagcttcccgtgagcggcggcagcagcactagcggcagccgccggagccgccgccgcagcggggacggggagccctcgggggccccaccgctgccgccgccgccacccgccgtcagctacccggactggatcggccagagttactccgaggtgatgagcctcaacgagcactcgatgcaggcgctgtcctggcgcaagctctacttaagccgcgccaagctcaaagcttccagccggacctcggctctgctctccggcttcgccatggtagcgatggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggccttctccacggtcatcgggacgctgcttttcctagcagaggtcgtgctgctctgctgggtcaagttcttacctctcaagaggcaagcgggacagccaagccccaccaagcctcccgctgaatcagtcatcgtcgccaaccacagcgacagcagcggcatcaccccgggtgaggcggcagccattgcctccaccgccatcatggttccctgtggcctggtttttatcgtctttgctgttcacttctaccgctccctggtcagccataagacggaccggcagttccaggagctcaatgagctggccgagtttgcccgcttgcaggaccagctggaccacagaggggaccattctctaacaccgggcacccactatgcctaagtcctcaccttcccactggccctttgaggccttggccttatgcccttctccatgaccttgtcctggcccagtccggaggacggcctgtgtaggcagctggctttgccagggcagagtgtggaaggaagaggctttaaaaaaaaaaaaatcactgtacttggagattttcttctgtgagaataagctttccctgttcttccagctgtctgtcccccctcctggtaggatgtggggggtggggcggggaatggggacagttgctctctttgtcccttcttctcccctgcaccagtgccacccggatgcttcctgtcctgccctcaaactccttactccttagccaagtgtgtgtgtgcgtgtgtgtgtgtgtgagagagaatatgtatgtgcgcacatgtctcctctctgtgcgtgtgtgtacatgcatgtgtgtgcaagtgtgcatgcacacttgtatgtacacataaaatatattcacaagggacacctcttcctcatgtctgtgtttgttgggtctagactgcctgatcggatggcccagtcaggtgagtcactaaacaccagtctttttaaaaattgagaggaaatggctcggggacaaaacactaacctgtcatggtcaaagccctaggattagtccaacagctgtaaaaaactgtgttgctggggctagaacccaggaatttgacaagcactctcccactgaattctactccaagacctgtaactttggaaaatttctactcaaaagaaaccttcagggatactaatt

SEQ ID NO: 5 - DNA, 21 bp, Artificial Sequence
  Primer
cccatatgtagatgtgttcag

SEQ ID NO: 6 - DNA, 20 bp, Artificial Sequence
  Primer
gagtgttgttcccttcacat

SEQ ID NO: 7 - DNA, 25 bp, Artificial Sequence
  Primer
ttatcgatgagcgtggtggttatgc

SEQ ID NO: 8 - DNA, 25 bp, Artificial Sequence
  Primer
gcgcgtacatcgggcaaataatatc

SEQ ID NO: 9 - DNA, 20 bp, Artificial Sequence
  Primer
cttggcctgggatctcagag

SEQ ID NO: 10 - DNA, 20 bp, Artificial Sequence
  Primer
tcagccattgccttcttgcc

SEQ ID NO: 11 - DNA, 20 bp, Artificial Sequence
  Primer
gcaggatctttagccagaag

SEQ ID NO: 12 - DNA, 20 bp, Artificial Sequence
  Primer
acatctgctgtcacgggtga